(19) United States
(12) Patent Application Publication
(10) Pub. No.: US 2023/0054069 A1
(43) Pub. Date: Feb. 23, 2023

(54) SYSTEMS AND METHODS FOR PREDICTING KIDNEY FUNCTION DECLINE

(71) Applicant: KLINRISK, INC., Wilmington, DE (US)

(72) Inventor: Navdeep Tangri, Winnipeg (CA)

(21) Appl. No.: 17/890,205

(22) Filed: Aug. 17, 2022

Related U.S. Application Data
(60) Provisional application No. 63/234,535, filed on Aug. 18, 2021.

(51) Int. Cl. G16H 50/50; G06N 20/20

(52) U.S. Cl. CPC G16H 50/50; G06N 20/20

(57) ABSTRACT

A method for generating a prediction of chronic kidney disease (CKD) progression includes accessing a machine learning model trained on a training dataset comprising (i) a first set of medical laboratory data associated with a plurality of patients, (ii) an age of each patient included in the plurality of patients, and (iii) a sex of each patient included in the plurality of patients. The first set of medical laboratory data indicates 20 medical measurements for at least a combination of patients included in the plurality of patients. The method further includes generating a prediction of CKD progression for a new patient by applying an input dataset associated with the new patient to the machine learning model. The input dataset includes an age and sex of the new patient and a second set of medical laboratory data indicating at least some of the 20 medical measurements for the new patient.

### CROSS-REFERENCE TO RELATED APPLICATIONS

This application claims priority to U.S. Provisional Patent Application No. 63/234,535, entitled “SYSTEMS AND METHODS FOR PREDICTING KIDNEY FUNCTION DECLINE” and filed on Aug. 18, 2021, which is incorporated herein by reference in its entirety.

### BACKGROUND

Chronic kidney disease (CKD) currently affects more than 850 million adults worldwide and is associated with increased morbidity and mortality and high health care costs. For instance, in 2009, the treatment of the end stage of CKD, e.g., kidney failure or end-stage renal disease (ESRD), required the expenditure of 40 billion dollars in the United States alone. Although only a few patients with CKD develop kidney failure, much of the excessive morbidity and costs associated with CKD are driven by individuals who progress to more advanced stages of CKD before reaching organ failure requiring dialysis.

Resource-efficient and appropriate treatment of patients with CKD serves to benefit the individuals affected by the disease and provides improved resource allocation in an increasingly burdened health care system. Accurate prediction of individual risk of CKD progression has the potential to improve patient experiences and outcomes through knowledge sharing and shared decision-making with patients, enhance care by better matching the risks and harms of therapy to the risk of disease progression, and/or improve health system efficiency by facilitating better alignment between resource allocation and individual risk.

Accordingly, there exists a need for improved techniques for predicting the risk of CKD progression for individuals.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIG. 1 illustrates an example computing environment that includes an example computing system that incorporates and/or is utilized to implement the disclosed embodiments.

FIG. 2 illustrates a conceptual representation of an example machine learning model trained on a training dataset comprising medical laboratory data and configured to generate a prediction of chronic kidney disease progression.

FIGS. 3A through 3D illustrates an example flow diagram depicting acts associated with generating a prediction of chronic kidney disease progression.

FIG. 4 illustrates an example report associated with a prediction of chronic kidney disease progression.

FIG. 5 schematically illustrates an example cohort of patients from which to generate a machine learning model training dataset.

FIG. 6A illustrates a table comprising a description of an example baseline cohort, including various test results to be included in the medical laboratory data for each patient.

FIG. 6B illustrates a table comprising an overview of variable missingness in the baseline cohort, as described in FIG. 6A.

FIG. 7 is an appendix of tariff codes used for defining dialysis and kidney transplantation.

FIG. 8 is a table that illustrates an overview of variable importance for each variable included in a machine learning model training dataset.

FIG. 9 illustrates a conceptual representation of an example training dataset comprising a 10 variable medical laboratory dataset.

FIG. 10 is a graph illustrating an example calibration plot for a machine learning model configured as a random forest model (e.g., using the training dataset as shown in FIG. 9 for a time period of two years).

FIG. 11 is a graph illustrating an example calibration plot for a machine learning model configured as a random forest model (e.g., using the training dataset as shown in FIG. 9 for a time period of five years).

FIG. 12 is a graph illustrating an example calibration plot for a machine learning model configured as a Cox model (e.g., using the training dataset as shown in FIG. 9 for a time period of two years).

FIG. 13 is a graph illustrating an example calibration plot for a machine learning model configured as a Cox model (e.g., using the training dataset as shown in FIG. 9 for a time period of five years).

FIG. 14 illustrates an example machine learning model trained on a training dataset comprising a 9 variable medical laboratory data and configured to generate a prediction of chronic kidney disease progression.

FIG. 15 is a graph illustrating an example calibration plot for a machine learning model configured as a Cox model, for example, using the training dataset as shown in FIG. 14, for a time period of two years.

FIG. 16 is a graph illustrating an example calibration plot for a machine learning model configured as a Cox model, for example, using the training dataset as shown in FIG. 14, for a time period of five years.

FIG. 17 illustrates an example training dataset comprising a 16 to 22 variable medical laboratory dataset.

FIG. 18 through 20 show graphs illustrating example calibration plots for machine learning models, for example, using the training dataset as shown in FIG. 17, for a time period of two years.

FIG. 21 illustrates an example training dataset comprising at least a 15 variable medical laboratory dataset.

FIG. 22 is a graph illustrating an example calibration plot for a machine learning model, for example, using the training dataset as shown in FIG. 21, for a time period of two years.

FIG. 23 is a graph illustrating an example calibration plot for a machine learning model, for example, using the training dataset as shown in FIG. 23, for a time period of five years.

FIG. 24 illustrates a table illustrating an example overview of performance evaluation statistics for various example machine learning models as disclosed herein and configured as Cox models.

FIG. 25 illustrates a calibration plot for various example machine learning models as disclosed herein and configured as Cox models.

FIGS. 26A and 26B illustrate tables showing various example overviews of performance evaluation statistics for various example machine learning models configured as random forest models.

FIG. 27A is a graph illustrating an example of a calibration plot for random forest models in subgroup analysis for patients with diabetes.

FIG. 27B is a graph illustrating an example of a calibration plot for random forest models in subgroup analysis for patients without diabetes.

FIGS. 27C-27D are graphs illustrating examples of a calibration plots for random forest models in subgroup analysis for patients with various stages of CKD.

FIG. 28 illustrates aspects of the validation cohort used to externally validate an example random survival forest model for generating CKD progression predictions.

FIG. 29 illustrates an overview of the degree of missingness for laboratory panels used to develop an example random survival forest model for generating CKD progression predictions.

FIG. 30 illustrates an overview of tariff codes for identifying dialysis and transplant for generating a training dataset for developing an example random survival forest model for generating CKD progression predictions.

FIG. 31 illustrates variable importance for an example 22-variable survival forest for generating CKD progression predictions.

FIG. 32 illustrates an overview of baseline descriptive statistics for a training cohort, an internal testing cohort, and an external validation cohort for developing an example random survival forest model for generating CKD progression predictions.

FIG. 33 illustrates AUC and Brier scores for years 1 through 5 for an example random survival forest model with 22 variables for generating CKD progression predictions.

FIG. 34 illustrates AUC and Brier scores for internal testing and external validation cohorts for an example random survival forest model with 22 variables for generating CKD progression predictions.

FIGS. 35A and 35B depict various calibration charts for an example random survival forest model with 22 variables for generating CKD progression predictions at 2 years.

FIG. 36 illustrates an overview of performance of an example random survival forest model with 22 variables for generating CKD progression predictions.

FIGS. 37A and 37B depict various calibration charts for an example random survival forest model with 22 variables for generating CKD progression predictions at 5 years.

FIG. 38 illustrates results of a heatmap model for generating CKD progression predictions.

FIG. 39 illustrates results of a clinical model for generating CKD progression predictions.

### DETAILED DESCRIPTION

Disclosed embodiments are directed to improved systems, methods, and/or frameworks for training and/or utilizing machine learning models to predict CKD progression and/or guide practitioners in care decisions for patients at risk of CKD progression.

The Kidney Failure Risk Equation (KFRE) is an internationally validated risk prediction that predicts the risk of progression to kidney failure for an individual patient with CKD. However, the KFRE has important limitations in that it applies only to later stages of CKD (G3-G5) and considers only the outcome of kidney failure requiring dialysis. In earlier stages of CKD, kidney failure is a rare event, even if progression to a more advanced stage is not. In these early stages, a decline in GFR of 40% is both clinically meaningful to patients and physicians and allows sponsors to design feasible randomized controlled trials at all stages of CKD.

In addition, new disease-modifying therapies for CKD that slow progression are available, but they have been largely studied in patients with preserved kidney function. Use of these therapies may be particularly beneficial in high-risk individuals with early stages of CKD where the benefit for dialysis prevention is large and cost-effectiveness may be achieved. Models for predicting a 40% decline in eGFR or the composite outcome of kidney failure or 40% decline in eGFR that can be applied to patients at all stages of CKD (G1-G5) may be implemented to apply disease-modifying therapies for CKD to high-risk individuals with early stages of CKD. When such models are based on laboratory data, they can be used through electronic health records or laboratory information systems, and are not subject to variability in coding, often found with CKD and its complications. At least some disclosed embodiments involve the derivation and external validation of new laboratory-based machine learning prediction models that accurately predict 40% decline in eGFR or kidney failure in patients (e.g., patients with CKD G1 to G5).

### Technical Benefits

The disclosed embodiments may facilitate various technical advantages over existing systems and methods associated with prediction of CKD progression, particularly in being able to predict chronic kidney disease progression for patients experiencing any stage of chronic kidney disease (CKD) (or patients with no CKD or unknown CKD status). Furthermore, predictions generated in accordance with the present disclosure may be based on a composite outcome of either 40% decline in eGFR and/or kidney failure (e.g., as opposed to solely kidney failure). Predictions generated in accordance with at least some embodiments of the present disclosure may provide a risk score for a patient experiencing either outcome.

In patients with CKD, the disclosed methods can be used to inform several important clinical decisions, such as, by way of non-limiting example: informing nephrology referral triage, evaluating the need for more intensive clinic care, determining the timing of modality education, dialysis access planning, and/or others. Disclosed embodiments for generating CKD progression predictions may be implemented in various ways, such as to generate CKD progression predictions for individual patients (e.g., when implemented in electronic health records or linked software solutions, and/or responsive to requests of individual physicians) and/or to facilitate batch processing of patients in patient databases (e.g., hospital or clinical databases).

At least some disclosed embodiments include models that predict individual outcomes (risk of 40% decline in eGFR or risk of kidney failure) or composite outcomes (risk of either kidney failure or 40% decline in eGFR occurring) that can be applied to patients screened for or at all stages of CKD (G1-G5). Systems and/or methods that provide such features are urgently needed. At least some models of the present disclosure may be utilized to risk stratify patients with early-stage disease (G1-G3) who are at high risk of CKD progression, inform enrollment of patients (at any CKD stage) in clinical trials, and/or guide implementation of therapies such as sodium-glucose cotransporter-2 (SGLT2) inhibitors or mineralocorticoid receptor antagonists (MRAs) that can modify disease progression.

### Systems and Techniques for Predicting CKD Progression

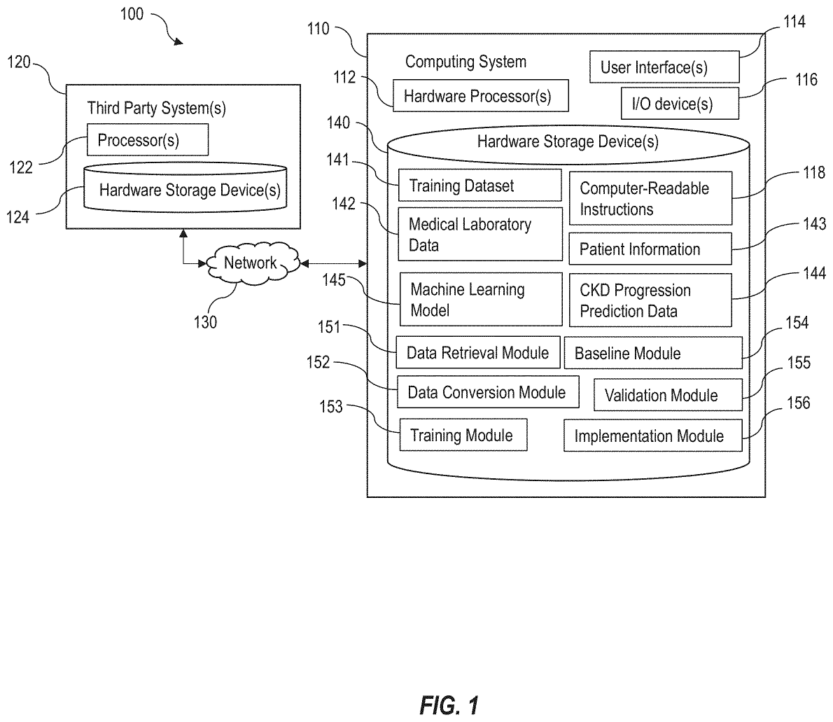

Attention will now be directed to FIG. 1, which illustrates example components of a computing system 110 which may include and/or be used to implement aspects of the disclosed invention. FIG. 1 depicts various machine learning (ML) modules and data types associated with inputs and outputs of the machine learning models.

As used herein, a machine learning model or module refers to any combination of software and/or hardware components that are operable to facilitate processing using machine learning models or other artificial intelligence-based structures/architectures. For example, one or more processors may comprise and/or utilize hardware components and/or computer-executable instructions operable to carry out function blocks and/or processing layers configured in the form of, by way of non-limiting example, random forest models, random survival forest models, Cox proportional hazards models, single-layer neural networks, feed forward neural networks, radial basis function networks, deep feed-forward networks, recurrent neural networks, long-short term memory (LSTM) networks, gated recurrent units, autoencoder neural networks, variational autoencoders, denoising autoencoders, sparse autoencoders, Markov chains, Hopfield neural networks, Boltzmann machine networks, restricted Boltzmann machine networks, deep belief networks, deep convolutional networks (or convolutional neural networks), deconvolutional neural networks, deep convolutional inverse graphics networks, generative adversarial networks, liquid state machines, extreme learning machines, echo state networks, deep residual networks, Kohonen networks, support vector machines, neural Turing machines, and/or others.

The example depicted in FIG. 1 illustrates the computing system 110 as part of a computing environment 100, which may include third-party system(s) 120 in communication (via a network 130) with the computing system 110. In some implementations, the computing system 110 is configured to train and/or configure a machine learning model (e.g., a CKD prediction model) to generate predictions of CKD progression for one or more patients. The machine learning model may additionally or alternatively be trained/configured to generate recommendations for treating, monitoring, or otherwise caring for the one or more patients. A computing system 110 of FIG. 1 may additionally or alternatively be configured to operate machine learning models, such as the CKD prediction model trained/configured as described herein.

The computing system 110 of FIG. 1 includes one or more processor(s) (such as one or more hardware processor(s)) 112 and storage (i.e., hardware storage device(s) 140) storing computer-readable instructions 118. The hardware storage device(s) 140 is/are able to house any number of data types and any number of computer-readable instructions 118 by which the computing system 110 is configured to implement one or more aspects of the disclosed embodiments when the computer-readable instructions 118 are executed by the one or more processor(s) 112. The hardware storage device(s) 140 may comprise physical, tangible storage means. The computing system 110 is also shown including user interface(s) 114 and input/output (I/O) device(s) 116.

As shown in FIG. 1, the hardware storage device(s) 140 is/are shown as a single storage unit. However, it will be appreciated that the hardware storage device(s) 140 may be implemented as a distributed storage that is distributed to several separate and sometimes remote systems and/or third-party system(s) 120. The computing system 110 can also comprise a distributed system with one or more of the components of computing system 110 being maintained/run by different discrete systems that may be remote from each other and that each perform different tasks. In some instances, a plurality of distributed systems performs similar and/or shared tasks for implementing the disclosed functionality, such as in a distributed cloud environment.

In the example of FIG. 1, the hardware storage device(s) 140 may store different data types including training dataset 141, medical laboratory data 142, patient information 143, and CKD progression prediction data 144. As shown in FIG. 1, the storage (e.g., hardware storage device(s) 140) may include computer-readable instructions 118, which may be usable to facilitate training/configuring and/or executing (e.g., for CKD progression prediction generation) of one or more of the models and/or modules shown in FIG. 1 (e.g., machine learning model 145).

The machine learning model 145 may be trained using a training dataset 141, which may comprise medical laboratory data (e.g., included in medical laboratory data 142) and/or other patient information (e.g., included in patient information 143) for a cohort of patients. The training dataset 141 may be applied to a machine learning model (e.g., machine learning model 145) to train the machine learning to generate a prediction of CKD progression. In some embodiments, the training dataset 141 comprises (i) a first set of medical laboratory data associated with a plurality of patients, (ii) an age of each patient included in the plurality of patients, and (iii) a sex of each patient included in the plurality of patients. The first set of medical laboratory data may include various labs/measurements associated with specific patients, such as, by way of non-limiting example, estimated glomerular filtration rate (eGFR), urine albumin-to-creatinine ratio (ACR), urea, serum sodium, serum chloride, serum hemoglobin, serum potassium, glucose, serum albumin, alkaline phosphatase, serum phosphate, serum bicarbonate, serum magnesium, serum calcium, aspartate aminotransferase (AST), alanine transaminase (ALT), bilirubin, gamma-glutamyl transferase (GGT), hematocrit, platelet count, and/or others.

The various labs/measurements associated with the various patients included in the training cohort may be collected (or have been collected) at one or more timepoints or during one or more time periods (e.g., resulting from samples or measurements obtained from each particular patient over the course of one or more patient-practitioner interactions over time, such as over the course of multiple sequential clinical appointments to obtain a series of samples or measurements over the course of a time period (e.g., a week, a month, etc.)). For example, several laboratory tests ordered on for a patient on a first day during a visit with a practitioner. As another example, a patient may provide one or more blood tests on a first day, and then submit a urine sample for testing on a different day. Alternatively, a particular test may require samples from multiple days over a time period of a week or a month, or even a year.

In some embodiments, a single time point is used for each set of lab values included in the training and/or testing data. For example, in some instances, a timepoint is defined by an eGFR lab measurement, where all other lab values are selected from labs within 365 days of the eGFR lab measurement.

The medical laboratory data 142 may be collected from patients based on one or more samples obtained from the patients at one or more single time periods (e.g., resulting from sample or measurements obtained from each particular patient during a respective single patient-practitioner interaction, such as during a single clinical appointment to obtain a single sample or measurement (e.g., a blood or urine sample)). The one or more samples may comprise various results from different blood, urine, and other lab tests.

In some implementations, the lab tests utilized to obtain the measurements represented in the training dataset 141 are routine lab tests that a patient typically has done during regular doctor office visits. For example, at least some of the measurements represented in the training dataset 141 may comprise one or more measurements obtained in association with a urine chemistry test (e.g., urine creatinine, urine albumin, urine ACR), a comprehensive metabolic panel (e.g., eGFR, glucose, calcium, sodium, albumin, potassium, bicarbonate, chloride, urea, phosphate/phosphorous, magnesium, liver enzymes), a complete blood cell count (e.g., hemoglobin, hematocrit, platelet count), a liver panel (e.g., ALT, AST, ALKP, GGT, bilirubin), and/or a uric acid test.

In some instances, one or more of the measurements represented in the training dataset 141 are derived or inferred from other measurements rather than being directly measured. For instance, a urine ACR measurement for a particular patient may be converted from a urine protein-to-creatinine test or a urine dipstick test.

It will be appreciated, in view of the present disclosure, that one or more measurements for one or more patients represented in the training dataset 141 may be missing or omitted from the training dataset 141. By way of non-limiting example, where a training dataset 141 includes medical laboratory data 142 for patient A and patient B, patient A may have labs/measurements that are unavailable for patient B, such as where a urine chemistry test and complete blood cell count were performed for both patient A and patient B, but a liver panel was only performed for patient A. Notwithstanding, the medical laboratory data 142 represented in the training dataset 141 may be regarded as including one or more measurements associated with a urine chemistry test, a complete blood cell count, and a liver panel, even where a liver panel was not obtained for patient B. In this regard, a set of labs/measurements may be represented in a training dataset 141 by a combination of patients (e.g., patient A and patient B) in the training cohort, even when one or more labs/measurements in the set of labs/measurements are missing for one or more patients in the combination of patients and even where no single patient exists in the training cohort for whom all of the labs/measurements of the set of labs/measurements are present (so long as each of the labs/measurements in the set of labs/measurements is included for at least one patient included in the training cohort).

In some implementations, the medical laboratory data 142 for the training dataset 141 has missing values for at least some patients represented in the medical laboratory data 142. In some instances, the training dataset 141 supplements missing values/measurements by utilizing imputed values, which may be imputed utilizing any suitable technique (e.g., adaptive tree imputation, proximity techniques, regression imputation, mean substitution, and/or others). For example, the training dataset 141 may include, for at its associated cohort of patients, eGFR, urine ACR, urea, potassium, hemoglobin, platelet count, albumin, calcium, glucose, bilirubin, sodium, bicarbonate, and/or GGT with a degree of value imputation of 30% or less (e.g., any of the foregoing measurements may comprise an imputed value for 30% or fewer of the patients in the cohort).

The training dataset 141 may include additional information associated with the plurality of patients (or cohort of patients), such as patient outcome information (e.g., included in patient information 143). Such patient outcome information may include whether and/or when the patients experienced a decline in eGFR (e.g., a 40% or other decline), kidney failure (e.g., necessitating dialysis or kidney transplant), and/or other clinical outcomes associated with CKD. The patient information 143 may additionally or alternatively comprise a stage of CKD of one or more patients. The stage of CKD may comprise stage G1, stage G2, stage G3, stage G4, or stage G5. The stage may, in some instances, also be selected from a plurality of sub-stages corresponding to each aforementioned stage (e.g., a substage of stage G1, etc.). The patient information 143 may also comprise the sex and/or gender of the patients, an age of the patients at the time of each sample collected from each of the patients, history of other diseases/medical conditions, family history of medical conditions, previous treatments/surgeries, and/or other relevant information such as blood pressure, temperature, oxygen levels, reflex tests, and/or other vitals. Such variables, however, are not necessary in certain embodiments and may be omitted.

The training dataset 141 may be utilized to train the machine learning model 145 in various ways (e.g., utilizing supervised learning techniques, unsupervised learning techniques, combinations thereof, and/or others). For instance, to build a random forest model, a system may build de-correlated trees by randomly sampling (e.g., bootstrap sampling) the original training dataset (e.g., training dataset 141), fitting a model to the randomly sampled (e.g., smaller) datasets, and aggregating the predictions. As another example, to build a random survival forest model, a system may randomly select subsets of features and/or thresholds for evaluation at each node for aggregation.

After the machine learning model 145 is trained, the machine learning model 145 may be utilized (run or executed) to generate predictions of CKD progression (e.g., CKD progression prediction data 144) for particular patients (e.g., for a new patient). For example, patient information (e.g., age and sex) may be obtained for a new patient in addition to medical laboratory data for the new patient. The medical laboratory data for the new patient may include one or more labs/measurements discussed hereinabove in association with the medical laboratory data 142 for the training dataset 141. For instance, the medical laboratory data for the new patient may comprise one or more of estimated glomerular filtration rate (eGFR), urine albumin-to-creatinine ratio (ACR), urea, serum sodium, serum chloride, serum hemoglobin, serum potassium, glucose, serum albumin, alkaline phosphatase, serum phosphate, serum bicarbonate, serum magnesium, serum calcium, aspartate aminotransferase (AST), alanine transaminase (ALT), bilirubin, gamma-glutamyl transferase (GGT), hematocrit, platelet count, and/or others. The labs/measurement for the new patient may include components of one or more of a urine chemistry test (e.g., urine creatinine, urine albumin, urine ACR), a comprehensive metabolic panel (e.g., eGFR, glucose, calcium, sodium, albumin, potassium, bicarbonate, chloride, urea, phosphate/phosphorous, magnesium, liver enzymes), a complete blood cell count (e.g., hemoglobin, hematocrit, platelet count), a liver panel (e.g., ALT, AST, ALKP, GGT, bilirubin), and/or a uric acid test.

The age, sex, and medical laboratory data for the new patient may be utilized as input to the (trained) machine learning model 145 to generate CKD progression prediction data 144 for the new patient. The CKD progression prediction data 144 may indicate a risk for the new patient to experience CKD progression, such as in the form of at least a 40% decline of eGFR. In some embodiments, the prediction of CKD progression additionally or alternatively indicates a risk of CKD progression in the form of kidney failure. For instance, the CKD progression prediction data 144 may indicate a risk of a composite CKD progression outcome occurring, where the composite outcome includes a 40% decline in eGFR or kidney failure (e.g., the patient experiencing a GFR of less than 10 ml/min/1.73 m², requiring chronic dialysis, or requiring a kidney transplant). As noted above, the machine learning model 145 may be utilized to generate such CKD progression prediction data 144 even for patients who are in early stages of CKD such as stage G1 or stage G2 or a substage thereof (e.g., for patients not in a CKD stage of G3 or later).

The prediction of CKD progression (e.g., CKD progression prediction data 144) may indicate a risk of experiencing CKD progression within a particular amount of time (e.g., from a timepoint associated with the input dataset for a new patient, such as a timepoint associated with an eGFR measurement for the new patient). By way of non-limiting example, the amount of time associated with the prediction of CKD progression may be 2 years, 5 years, or another amount of time (e.g., 6 months, one year, 18 months, 3 years, 4 years, etc.).

In some implementations, separate machine learning models 145 (e.g., separate random forest models) are trained for generating CKD progression predictions associated with different time horizons (e.g., one model for 2-year CKD progression predictions, a separate model for 5-year CKD progression predictions, etc.). In some implementations, a single machine learning model 145 (e.g., a single random survival forest model) is trained for generating CKD progression predictions associated with different time horizons. For instance, a time horizon or particular amount of time (e.g., 2 years, 5 years, or any amount of time or number of days) may be provided as input to the machine learning model 145 in combination with the sex, age, and medical laboratory data for a new patient to cause the machine learning model 145 to generate a prediction of CKD progression for the input time horizon or particular amount of time.

FIG. 1 further illustrates additional example modules which may be stored on hardware storage device(s) 140 and/or otherwise associated with the computing system 110. The additional modules may include or more of a data retrieval module 151, a data conversion module 152, a training module 153, a validation module 155, and/or an implementation module 156.

As used herein, the term “module” can refer to any combination of hardware components or software objects, routines, or methods that may configure a computing system 110 to carry out certain acts. For instance, the different components, modules, engines, devices, and/or services described herein may be implemented utilizing one or more objects or processors that execute on computing system 110 (e.g., as separate threads). While FIG. 1 depicts several independent modules, one will understand the characterization of a module is at least somewhat arbitrary. In at least one implementation, the various modules described herein may be combined, divided, or excluded in configurations other than that which is explicitly described or illustrated. For example, any of the functions described herein with reference to any particular module may be performed utilizing any number and/or combination of processing units, software objects, modules, instructions, computing centers (e.g., computing centers that are remote to computing system 110), etc. In the present description, the individual modules are provided for the sake of clarity and explanation and are not intended to be limiting.

The data retrieval module 151 can be configured to locate and access data sources, databases, and/or storage devices comprising one or more data types from which the data retrieval module 151 can extract sets or subsets of data to be used as training data. The data retrieval module 151 can receive data from the databases and/or hardware storage devices, wherein the data retrieval module 151 is configured to reformat or otherwise modify the received data to be used as training data. Additionally, or alternatively, the data retrieval module 151 can be in communication with one or more remote systems (e.g., third-party system(s) 120) comprising third-party datasets and/or data sources. In some instances, these data sources comprise patient laboratory test results and other patient information portals.

The data retrieval module 151 can access electronically stored information comprising medical laboratory data 142, patient information 143, and/or CKD progression prediction data 144. The data retrieval module 151 can be configured as a smart module that is able to learn optimal dataset extraction processes to obtain a sufficient amount of data in a timely manner as well as retrieve data that is most applicable to the desired applications for which the machine learning models/modules will be trained. For example, the data retrieval module 151 can learn which databases and/or datasets will generate training data that will train a model (e.g., for a specific query or specific task) to increase accuracy, efficiency, and/or efficacy of that model in the desired chronic kidney disease prediction techniques.

The data retrieval module 151 can locate, select, and/or store raw recorded source data when the data retrieval module 151 is in communication with one or more ML module(s) and/or models included in computing system 110. In such instances, the other modules in communication with the data retrieval module 151 can receive data that has been retrieved (i.e., extracted, pulled, etc.) from one or more data sources such that the received data is further augmented and/or applied to downstream processes. For example, the data retrieval module 151 can be in communication with the training module 153 and/or implementation module 156. The data retrieval module 151 may be configured to retrieve training datasets (e.g., training dataset 141) comprising the medical laboratory data 142 and patient information 143.

In some instances, the data conversion module 152 is configured to convert any raw data retrieved by the data retrieval module 151 into workable data to be included in the training dataset 141.

In some instances, the training module 153 is in communication with one or more of the data retrieval module 151, the data conversion module 152, the validation module 154 and/or the implementation module 156. In such embodiments, the training module 153 is configured to receive one or more training datasets (e.g., training dataset 141) via the data retrieval module 151. After receiving training data relevant to a particular application or task, the training module 153 may train one or more models on the training data. The training module 153 can be configured to train a model via unsupervised training and/or supervised training. The training module 153 is configured to train a machine learning model 145 to generate a prediction of chronic kidney disease progression by applying a training dataset 141 comprising medical laboratory data 142 and patient information 143 in order to produce as output the CKD progression prediction data 144.

In some embodiments, the training dataset 141 is split into a training dataset and a validation dataset. The validation module 155 is configured to utilize the validation dataset to test the machine learning model 145 for accuracy and precision in predicting CKD progression. For example, a random forest model can be fit using the Random Forest for Survival, Regression and Classification (RF-SRC) package in R using any desired demographic and laboratory variables. For instance, available data can be split into training (e.g., 70%) and testing/validation (e.g., 30%) datasets. The parameters could include a node size of 15 (or other size), and the number of trees equal to 60 (or other number of trees). Additional or alternative random forest or random survival forest (or other) models may be used within the scope of the present disclosure.

The computing system 110 includes an implementation module 156 in communication with any one of the models and/or ML model 145 (or all the models/modules) included in the computing system 110 such that the implementation module 156 is configured to implement, initiate, or run one or more functions of the modules. In one example, the implementation module 156 is configured to operate the data retrieval modules 151 so that the data retrieval module 151 retrieves data at the appropriate time to be able to generate training data for the training module 153. The implementation module 156 can facilitate the process communication and timing of communication between one or more of the modules and may configured to implement and/or operate a machine learning model 145 which is configured as a CKD progression prediction model.

The computing system can be in communication with third-party system(s) 120 comprising one or more processor(s) 122, one or more of the computer-readable instructions 118, and one or more hardware storage device(s) 124. The third-party system(s) 120 may further comprise databases housing data that could be used as training data, for example, medical laboratory data not included in local storage. Additionally, or alternatively, the third-party system(s) 120 include machine learning systems external to the computing system 110.

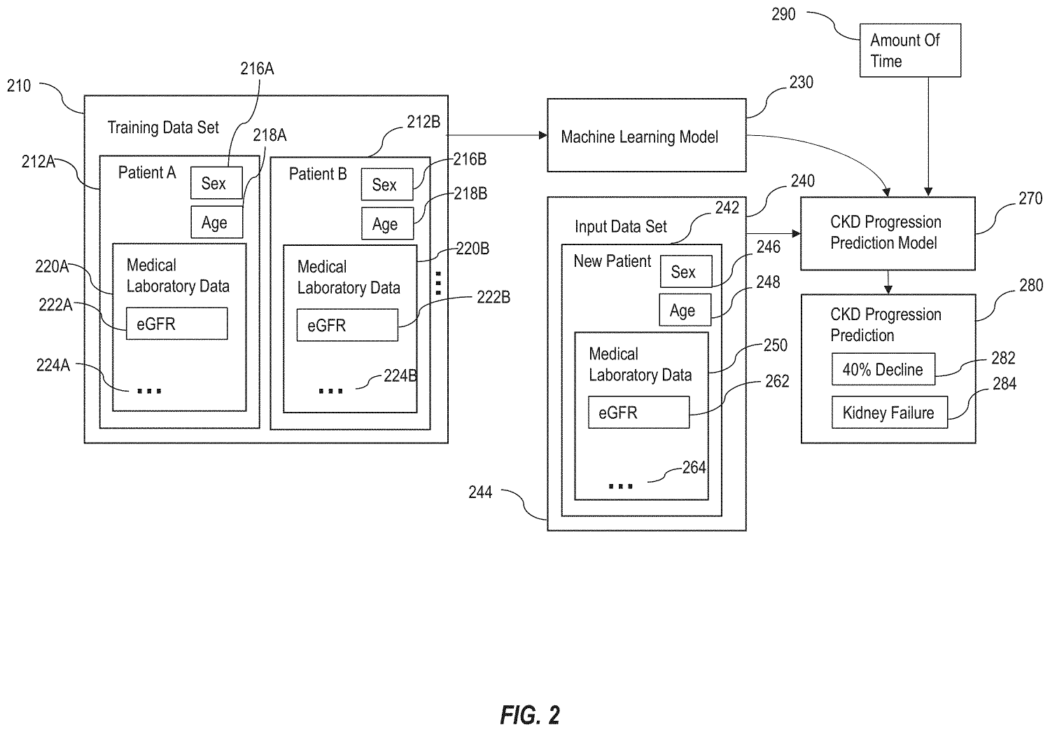

FIG. 2 illustrates an example machine learning model 230 (e.g., machine learning model 145 of FIG. 1) trained on a training data set 210 (e.g., training dataset 141) comprising medical laboratory data 220A/220B (e.g., medical laboratory data 142) and patient information (e.g., patient information 143) comprising a CKD stage 214A/214B, a sex 216A/216B, and an age 218A/218B for a plurality of patients (e.g., patient A 212A and patient B 212B). The machine learning model 230 is configured to generate a prediction of chronic kidney disease progression 280 (e.g., CKD progression prediction data 144) for a new patient 242. The medical laboratory data 220A comprises at least an eGFR 222A for patient A and may comprise additional labs/measurements for patient A (as indicated by ellipsis 224A). Similarly, medical laboratory data 220B comprises at least an eGFR 222B for patient B and may comprise additional labs/measurements for patient B (as indicated by ellipsis 224B). The training data set 210 comprises data for any number of patients (as indicated in FIG. 2 by the ellipsis associated with the training data set 210).

The training data set 210 is then applied to the machine learning model 230 to train the machine learning model 230 to generate a prediction of CKD progression, thereby providing a CKD progression prediction model 270. A new input data set 240 associated with a new patient 242 (e.g., a patient not included in the training data set 210, or a patient for whom a prediction of CKD progression is desired) is applied as input to the CKD progression prediction model 270 to generate a CKD progression prediction 280 for the new patient 242. The input data set 242 comprises a CKD stage 244, a sex 246, an age 248 and medical laboratory data 250 for the new patient. The medical laboratory data 250 (for the new patient 242) comprises at least an eGFR 262 based on one or more samples obtained from the new patient (e.g., at a single timepoint or single time period resulting from samples and/or information obtained from/about the new patient within a single patient-practitioner appointment, within a single day, within a single hour, etc.). The medical laboratory data 250 for the new patient 242 may additionally comprise one or more other labs/measurements (as indicated by ellipsis 264). The CKD progression prediction 280 comprises a risk score for the new patient experiencing a 40% decline in the eGFR 282 and/or kidney failure 284 within a designated timeframe (e.g., within 2 years or within 5 years).

As noted above, the timeframe or particular amount of time 290 associated with the CKD progression prediction 280 may be provided as input to the CKD progression prediction model 270, such as where the CKD progression prediction model 270 is implemented as a random survival forest model. In some instances, an input timeframe or particular amount of time 290 is not provided as an input, and instead the CKD progression prediction model 270 is selected from a plurality of CKD progression prediction models, each being associated with a different timeframe or particular amount of time.

The following discussion now refers to a number of methods (e.g., computer-implementable or system-implementable methods) and/or method acts that may be performed in accordance with the present disclosure. Although the method acts are discussed in a certain order and are illustrated in a flow chart as occurring in a particular order, no particular ordering is required unless specifically stated, or required because an act is dependent on another act being completed prior to the act being performed. One will appreciate that certain embodiments of the present disclosure may omit one or more of the acts described herein. The various acts described herein may be performed utilizing one or more computing system components described hereinabove (e.g., hardware processor(s) 112, hardware storage device(s) 140, instructions and/or modules, etc.).

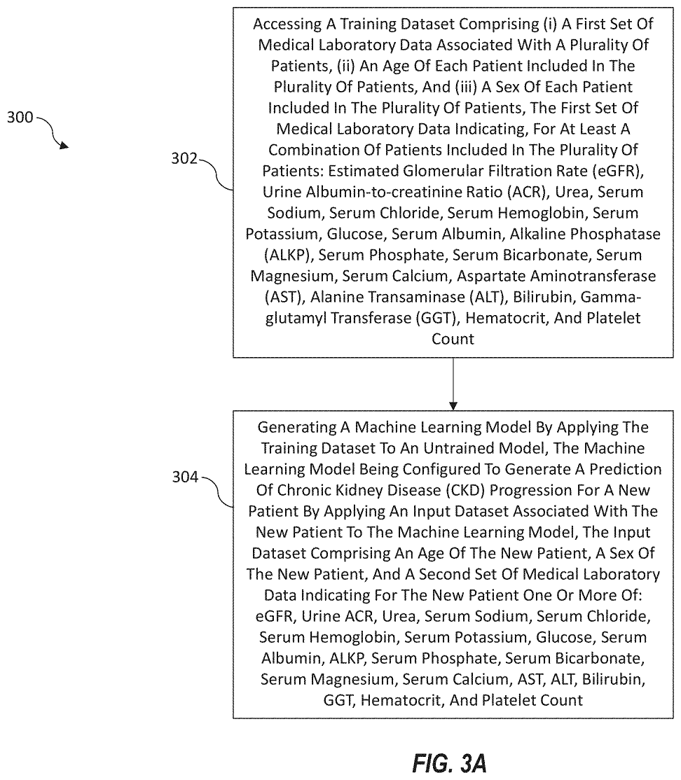

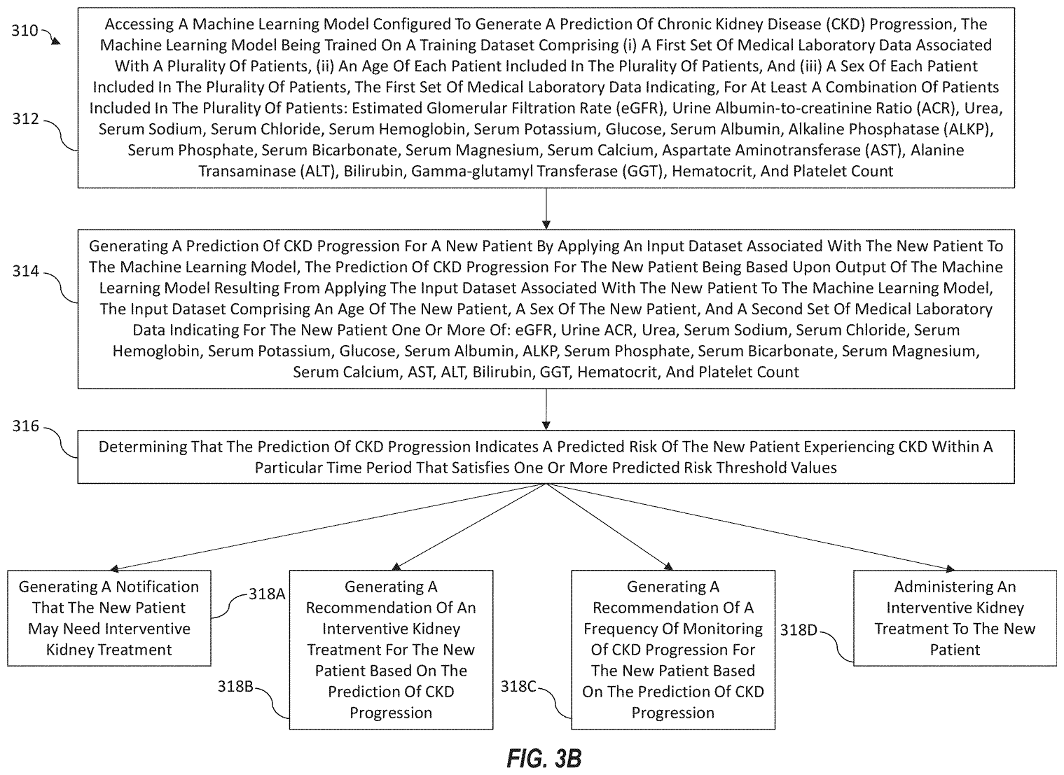

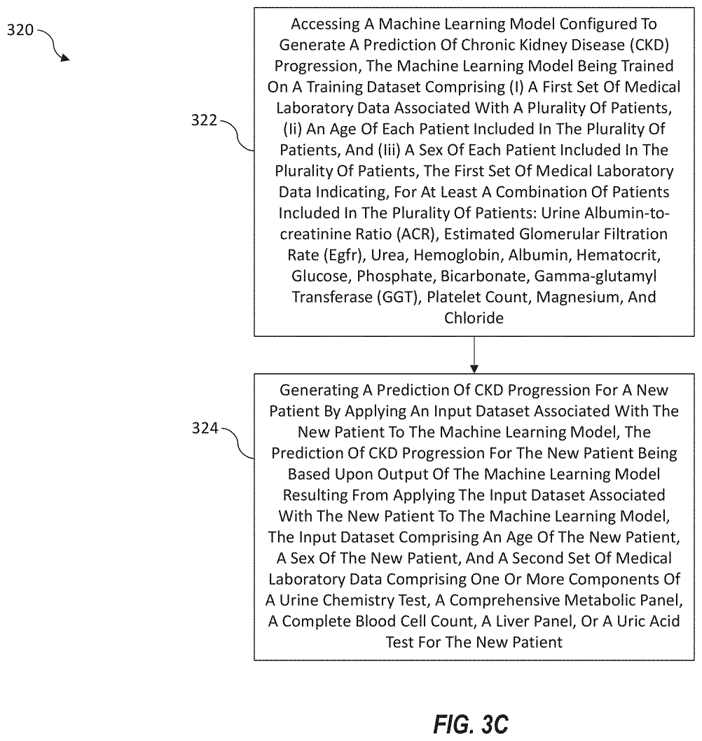

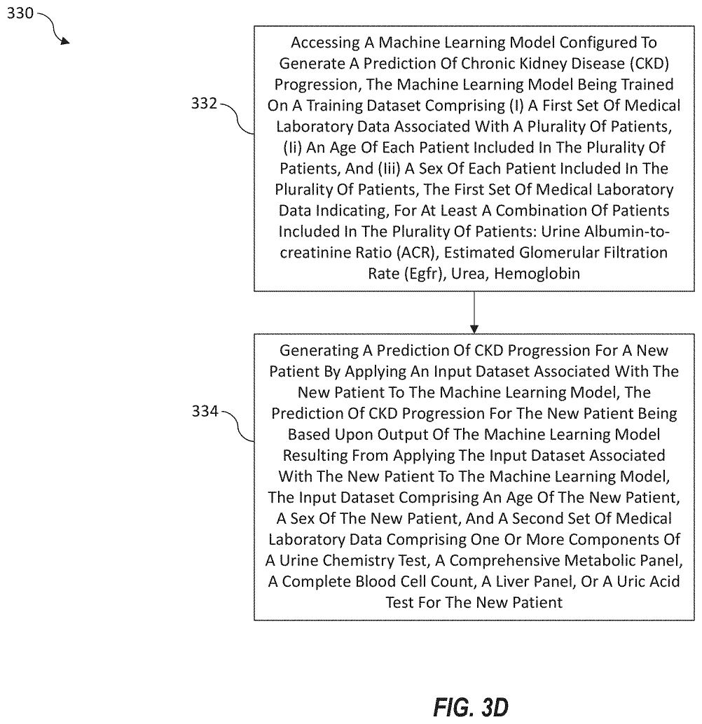

FIG. 3A illustrates an example flow diagram 300 depicting acts associated with generating a machine learning model for predicting CKD progression.

Act 302 of flow diagram 300 includes accessing a training dataset comprising (i) a first set of medical laboratory data associated with a plurality of patients, (ii) an age of each patient included in the plurality of patients, and (iii) a sex of each patient included in the plurality of patients, the first set of medical laboratory data indicating, for at least a combination of patients included in the plurality of patients: estimated glomerular filtration rate (eGFR), urine albumin-to-creatinine ratio (ACR), urea, serum sodium, serum chloride, serum hemoglobin, serum potassium, glucose, serum albumin, alkaline phosphatase (ALKP), serum phosphate, serum bicarbonate, serum magnesium, serum calcium, aspartate aminotransferase (AST), alanine transaminase (ALT), bilirubin, gamma-glutamyl transferase (GGT), hematocrit, and platelet count.

Act 304 of flow diagram 300 includes generating a machine learning model by applying the training dataset to an untrained model, the machine learning model being configured to generate a prediction of chronic kidney disease (CKD) progression for a new patient by applying an input dataset associated with the new patient to the machine learning model, the input dataset comprising an age of the new patient, a sex of the new patient, and a second set of medical laboratory data indicating for the new patient one or more of: eGFR, urine ACR, urea, serum sodium, serum chloride, serum hemoglobin, serum potassium, glucose, serum albumin, ALKP, serum phosphate, serum bicarbonate, serum magnesium, serum calcium, AST, ALT, bilirubin, GGT, hematocrit, and platelet count.

One will appreciate, in view of the present disclosure, that the medical laboratory data utilized as input to the machine learning model can take on various forms, and that the machine learning model may treat the input data in various ways. For instance, any of the measurements may comprise continuous measurements, categorical measurements, transformed/modified measurements (e.g., log-transformed measurements), mathematically modified measurements (e.g., squared, cubed, etc.), etc.

In some instances, the machine learning model comprises a random survival forest model configured to receive time period input (e.g., a number of days, months, years, etc.) in addition to the input dataset to generate the prediction of CKD progression for the input time period (e.g., a likelihood of experiencing CKD progression such as 40% decline in eGFR and/or kidney failure within the input time period). In some instances, the machine learning model comprises a random forest model configured to generate a prediction CKD progression for a particular time period. Multiple models may be generated for generating CKD progression predictions for different time horizons.

FIGS. 3B through 3D illustrate an example flow diagrams 310, 320, and 330, respectively, depicting acts associated with generating predictions of CKD progression for new patients.

Act 312 of flow diagram 310 of FIG. 3B includes accessing a machine learning model configured to generate a prediction of chronic kidney disease (CKD) progression, the machine learning model being trained on a training dataset comprising (i) a first set of medical laboratory data associated with a plurality of patients, (ii) an age of each patient included in the plurality of patients, and (iii) a sex of each patient included in the plurality of patients, the first set of medical laboratory data indicating, for at least a combination of patients included in the plurality of patients: estimated glomerular filtration rate (eGFR), urine albumin-to-creatinine ratio (ACR), urea, serum sodium, serum chloride, serum hemoglobin, serum potassium, glucose, serum albumin, alkaline phosphatase (ALKP), serum phosphate, serum bicarbonate, serum magnesium, serum calcium, aspartate aminotransferase (AST), alanine transaminase (ALT), bilirubin, gamma-glutamyl transferase (GGT), hematocrit, and platelet count.

In some implementations, the machine learning model comprises a random survival forest model. The first set of medical laboratory data may comprise one or more imputed values in place of missing values. In some instances, the first set of medical laboratory data indicates, with a degree of value imputation of 30% or less, eGFR, urine ACR, urea, potassium, hemoglobin, platelet count, albumin, calcium, glucose, bilirubin, sodium, bicarbonate, and GGT.

Act 314 of flow diagram 310 includes generating a prediction of CKD progression for a new patient by applying an input dataset associated with the new patient to the machine learning model, the prediction of CKD progression for the new patient being based upon output of the machine learning model resulting from applying the input dataset associated with the new patient to the machine learning model, the input dataset comprising an age of the new patient, a sex of the new patient, and a second set of medical laboratory data indicating for the new patient one or more of: eGFR, urine ACR, urea, serum sodium, serum chloride, serum hemoglobin, serum potassium, glucose, serum albumin, ALKP, serum phosphate, serum bicarbonate, serum magnesium, serum calcium, AST, ALT, bilirubin, GGT, hematocrit, and platelet count. As used herein, “urine ACR” may comprise a direct urine ACR measurement, a derived or estimated urine ACR, and/or components of urine ACR such as urine albumin, urine creatinine, urine protein, and/or qualitative urine albumin (e.g., from dipstick).

In some instances, the new patient is not associated with a CKD stage of G3 or later. In some implementations, the prediction of CKD progression comprises a prediction of a risk of the new patient experiencing kidney failure or about a 40% or greater decline of the eGFR for the new patient. In some instances, the risk of kidney failure comprises an indication that the new patient is at risk of (i) requiring chronic dialysis, (ii) requiring a kidney transplant, or (iii) experiencing a glomerular filtration rate of less than 10 ml/min/1.73 m².

The prediction of CKD progression may indicate a risk of experiencing CKD progression within a particular amount of time from a time period associated with the input dataset for the new patient (e.g., an amount of time from an eGFR measurement associated with the new patient). In some implementations, such as where the machine learning model is implemented as a random survival forest model, the particular amount of time is provided as input to the machine learning model for generating the prediction of CKD progression. The particular amount of time may comprise 2 years 5 years, or any amount of time.

The urine ACR for one or more of the plurality of patients or the new patient may be converted from a urine protein-to-creatinine test or a urine dipstick test.

Act 316 of flow diagram 310 includes determining that the prediction of CKD progression indicates a predicted risk of the new patient experiencing CKD within a particular time period that satisfies one or more predicted risk threshold values. The one or more predicted risk threshold values may be based upon the particular time period associated with the prediction of CKD progression (e.g., different time horizons may have different sets of thresholds). In one example, for a 2 year time period, a 2% or greater prediction of CKD progression (e.g., indicating a 2% likelihood that the new patient experiences CKD progression in the form of a 40% reduction in eGFR or kidney failure is 2%) may be associated with an “intermediate” risk classification for the new patient and a 10% or greater prediction of CKD progression may be associated with a “high” risk classification for the new patient. As another example, for a 5 year time period, a 5% or greater prediction of CKD progression may be associated with an “intermediate” risk classification for the new patient and a 25% or greater prediction of CKD progression may be associated with a “high” risk classification for the new patient. Additional or alternative threshold structures for the same or different time horizons are within the scope of the present disclosure.

One or more of acts 318A through 318D may be performed based upon performance of act 316. Act 318A includes generating a notification that the new patient may need interventive kidney treatment. Act 318B includes generating a recommendation of an interventive kidney treatment for the new patient based on the prediction of CKD progression. Act 318C includes generating a recommendation of a frequency of monitoring of CKD progression for the new patient based on the prediction of CKD progression. Act 318D includes administering an interventive kidney treatment to the new patient. The acts 318A, 318B, 318C, and/or 318D performed responsive to the prediction of CKD progression satisfying the one or more thresholds in accordance with act 316 may be selected based upon the particular time period associated with the prediction of CKD progression (e.g., 2 year or 5 year), the particular threshold(s) satisfied (e.g., whether the patient is classified as being at “intermediate” or “high” risk), and/or one or more other factors such as at least some of the set of laboratory for the new patient (e.g., used as part of the input dataset for generating the prediction of CKD progression for the new patient).

Various illustrative examples associated with acts 318A through 318D will now be discussed. In some instances, performance of act 318A may include generating a notification of complications that may arise associated with CKD for the new patient, which may be based on individualized patient labs/measurements and/or other patient data for the new patient.

For example, in response to determining that the new patient is a man with a hemoglobin less than about 130 g/L or a woman with a hemoglobin of less than about 120 g/L, act 318A may involve generating a notification indicating that anemia is a potential complication for the new patient.

As another example, in response to determining that the new patient has a potassium greater than about 5 mEq/L, act 318A may involve generating a notification indicating that hyperkalemia is a potential complication for the new patient.

As another example, in response to determining that the new patient has a serum bicarbonate less than about 22 mEq/L, act 318A may involve generating a notification indicating that metabolic acidosis is a potential complication for the new patient.

As another example, in response to determining that the new patient has a phosphorus of greater than about 1.6 mg/dL and/or a calcium less than about 2.1 millimoles/L or greater than about 2.7 millimoles/L, act 318A may involve generating a notification indicating that CKD mineral bone disease (CKD-MBD) is a potential complication for the new patient.

In some instances, the recommendations generated in accordance with act 318B may be based on individualized patient labs/measurements and/or other patient data for the new patient, and/or based on the complications noted above with respect to act 318A.

For example, in response to determining that the new patient has an age greater than about 50 and has an eGFR of less than about 60 mL/min/1.73 m² or a urine ACR greater than about 3 mg/mmol, act 318B may involve generating a recommendation that the new patient be prescribed statins (and/or other cholesterol treatments).

As another example, in response to determining that the new patient has an eGFR of less than about 30 mL/min/1.73 m² and has been classified as being at “high” risk of CKD progression in accordance with act 316, act 318B may involve generating a recommendation that the new patient be referred to nephrology.

As another example, in response to determining that the new patient has been classified as being at “intermediate” or “high” risk of CKD progression in accordance with act 316, act 318B may involve generating a recommendation that the new patient undergo renin-angiotensin-aldosterone system (RAAS) inhibition (e.g., unless the new patient has a potassium greater than about 5 mEq/L or an eGFR of less than about 15 mL/min/1.73 m²; RAAS inhibition may be strongly recommended if the new patient has an eGFR of greater than about 15 mL/min/1.73 m² and a urine ACR greater than about 3 mg/mmol), non-steroidal mineralocorticoid receptor antagonists (MRAs) therapy (e.g., unless the new patient has a potassium greater than about 5 mEq/L or an eGFR of less than about 25 mL/min/1.73 m²; 10 mg per day may be recommended if the new patient has an eGFR within a range of about 25 mL/min/1.73 m² to about 60 mL/min/1.73 m²; 20 mg per day may be recommended if the new patient has an eGFR greater than about 60 mL/min/1.73 m²), and/or sodium-glucose cotransporter-2 (SGLT2) inhibitor medication (e.g., unless the new patient has an eGFR of less than about 20 mL/min/1.73 m²).

As another example, in response to determining that anemia is a potential complication for the new patient (as discussed above with reference to act 318A), act 318B may involve generating a recommendation that iron studies such as ferritin, serum iron, and/or total iron binding capacity (TIBC) be obtained for the new patient (e.g., at regular monitoring intervals, such as those discussed hereinbelow with reference to act 318C).

As another example, in response to determining that hyperkalemia is a potential complication for the new patient (as discussed above with reference to act 318A), act 318B may involve generating a recommendation that the patient undergo a low potassium diet (e.g., if the new patient has a potassium within a range of about 5 mEq/L to about 5.5 mEq/L) and/or receive hyperkalemia monitoring and/or treatment in accordance with clinical practice guidelines (e.g., if the new patient has a potassium greater than about 5.5 mEq/L).

As another example, in response to determining that metabolic acidosis is a potential complication for the new patient (as discussed above with reference to act 318A), act 318B may involve generating a recommendation that the patient undergo metabolic acidosis monitoring and/or treatment in accordance with clinical practice guidelines.

As another example, in response to determining that CKD-MBD is a potential complication for the new patient (as discussed above with reference to act 318A), act 318B may involve generating a recommendation that the patient undergo a low phosphorus diet.

In some instances, act 318B may comprise recommending one or more blood pressure targets for the new patient, such as a target blood pressure of about 130/80 mm Hg (or a target systolic blood pressure of about 120 mm Hg if the new patient has an eGFR of less then about 60 mL/min/1.73 m² or a urine ACR greater than about 3 mg/mmol).

In some instances, the recommendations generated in accordance with act 318C may be based on individualized patient labs/measurements and/or other patient data for the new patient, and/or based on the complications noted above with respect to act 318A.

For example, in response to determining that the new patient has been classified as being at “high” risk of CKD progression in accordance with act 316 and has an eGFR of less than about 60 mL/min/1.73 m², act 318C may involve generating a recommendation that the new patient undergo CKD monitoring at least four times per year (or more).

As another example, in response to determining that the new patient has been classified as being at “high” risk of CKD progression in accordance with act 316 and has an eGFR of greater than about 60 mL/min/1.73 m², act 318C may involve generating a recommendation that the new patient undergo CKD monitoring three times per year (or more).

As another example, in response to determining that the new patient has been classified as being at “intermediate” risk of CKD progression in accordance with act 316 and has an eGFR of less than about 45 mL/min/1.73 m², act 318C may involve generating a recommendation that the new patient undergo CKD monitoring three times per year (or more).

As another example, in response to determining that the new patient has been classified as being at “intermediate” risk of CKD progression in accordance with act 316 and has an eGFR of greater than about 45 mL/min/1.73 m², act 318C may involve generating a recommendation that the new patient undergo CKD monitoring two times per year (or more).

As another example, in response to determining that the new patient has been classified as being at “low” risk of CKD progression in accordance with act 316 (e.g., the new patient is not classified as “intermediate” or “high” risk), act 318C may involve generating a recommendation that the new patient undergo CKD monitoring one time per year (or more).

Act 318D may comprise carrying out one or more of the recommendations discussed above with reference to acts 318B and/or 318C (e.g., RAAS inhibition, blood pressure control, SGLT2 inhibitor medication, MRAs therapy) and/or others (e.g., preparation for nephrology consultation, home dialysis, and/or kidney transplant).

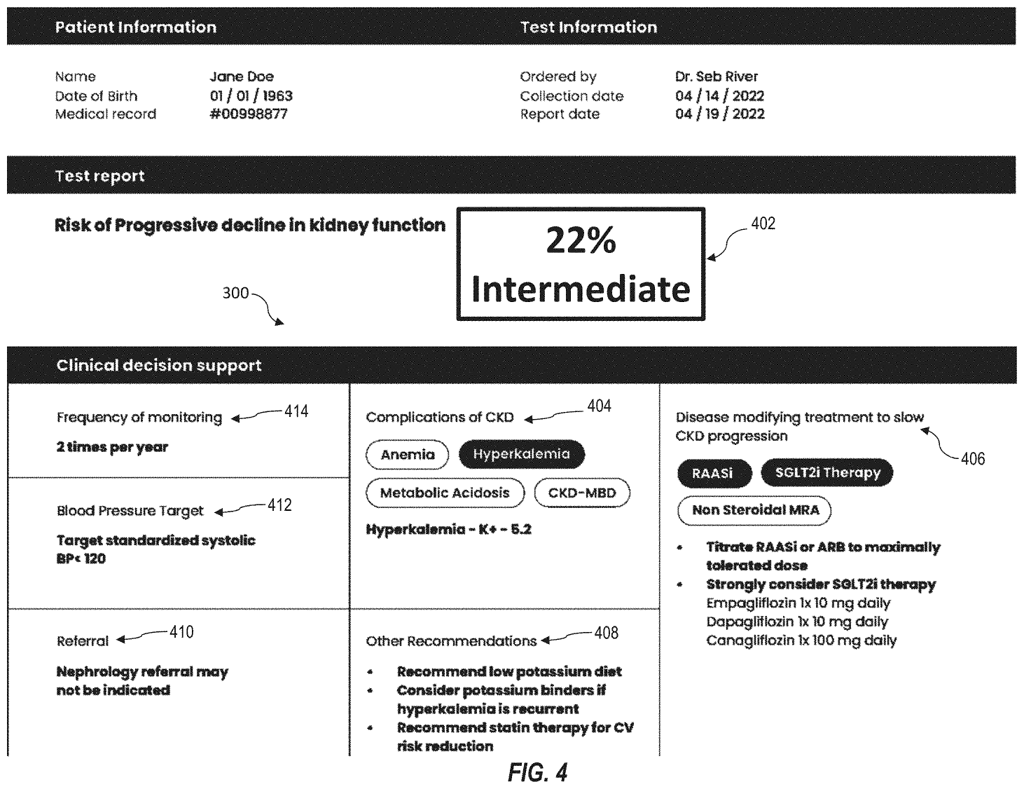

FIG. 4 illustrates an example report that includes various components discussed hereinabove with reference to acts 314, 316, 318A, 318B, and/or 318C, such as a prediction of CKD progression 402 (indicating a 22% risk of CKD progression for a 5 year time horizon, which is characterized as “intermediate” based on satisfying a threshold of being over 5% but less than 25%), potential complications of CKD 404, recommended treatments 406 and additional recommendations 408, a nephrology referral recommendation 410, a blood pressure target recommendation 412, and a monitoring frequency recommendation 414.

A report similar (in at least some respects) to that shown in FIG. 4 may be generated responsive to a request made by a physician or in accordance with implemented primary care practices (e.g., as a routine practice for patients meeting certain criteria). One will appreciate, in view of the present disclosure, that a report in accordance with the present disclosure may include additional or alternative components and may take on various forms/formats.

Attention is directed to FIG. 3C, which illustrates that act 322 of flow diagram 320 includes accessing a machine learning model configured to generate a prediction of chronic kidney disease (CKD) progression, the machine learning model being trained on a training dataset comprising (i) a first set of medical laboratory data associated with a plurality of patients, (ii) an age of each patient included in the plurality of patients, and (iii) a sex of each patient included in the plurality of patients, the first set of medical laboratory data indicating, for at least a combination of patients included in the plurality of patients: urine albumin-to-creatinine ratio (ACR), estimated glomerular filtration rate (eGFR), urea, hemoglobin, albumin, hematocrit, glucose, phosphate, bicarbonate, gamma-glutamyl transferase (GGT), platelet count, magnesium, and chloride.

Act 324 of flow diagram 320 includes generating a prediction of CKD progression for a new patient by applying an input dataset associated with the new patient to the machine learning model, the prediction of CKD progression for the new patient being based upon output of the machine learning model resulting from applying the input dataset associated with the new patient to the machine learning model, the input dataset comprising an age of the new patient, a sex of the new patient, and a second set of medical laboratory data comprising one or more components of a urine chemistry test, a comprehensive metabolic panel, a complete blood cell count, a liver panel, or a uric acid test for the new patient.

In some implementations, the second set of medical laboratory data comprises one or more components of the urine chemistry test, the comprehensive metabolic panel, and the complete blood cell count for the new patient. Although not shown in FIG. 3C, flow diagram 320 may further include acts similar to acts 316, 318A, 318B, 318C, and/or 318D for performance based on the prediction of CKD progression generated in accordance with act 324.

Act 332 of flow diagram 330 of FIG. 3D includes accessing a machine learning model configured to generate a prediction of chronic kidney disease (CKD) progression, the machine learning model being trained on a training dataset comprising (i) a first set of medical laboratory data associated with a plurality of patients, (ii) an age of each patient included in the plurality of patients, and (iii) a sex of each patient included in the plurality of patients, the first set of medical laboratory data indicating, for at least a combination of patients included in the plurality of patients: urine albumin-to-creatinine ratio (ACR), estimated glomerular filtration rate (eGFR), urea, hemoglobin.

Act 334 of flow diagram 330 includes generating a prediction of CKD progression for a new patient by applying an input dataset associated with the new patient to the machine learning model, the prediction of CKD progression for the new patient being based upon output of the machine learning model resulting from applying the input dataset associated with the new patient to the machine learning model, the input dataset comprising an age of the new patient, a sex of the new patient, and a second set of medical laboratory data comprising one or more components of a urine chemistry test, a comprehensive metabolic panel, a complete blood cell count, a liver panel, or a uric acid test for the new patient.

In some implementations, the second set of medical laboratory data comprises one or more components of the urine chemistry test for the new patient. In some instances, the second set of medical laboratory data comprises one or more components of the urine chemistry test and the comprehensive metabolic panel for the new patient. Although not shown in FIG. 3D, flow diagram 330 may further include acts similar to acts 316, 318A, 318B, 318C, and/or 318D for performance based on the prediction of CKD progression generated in accordance with act 334.

As noted hereinabove, various types of machine learning models may be implemented to facilitate generation of predictions of CKD progression for patients in accordance with the present disclosure. The following discussion refers to example implementations of various random forest models and random survival forest models for generating predictions of CKD progression.

### Random Forest Model Example(s)

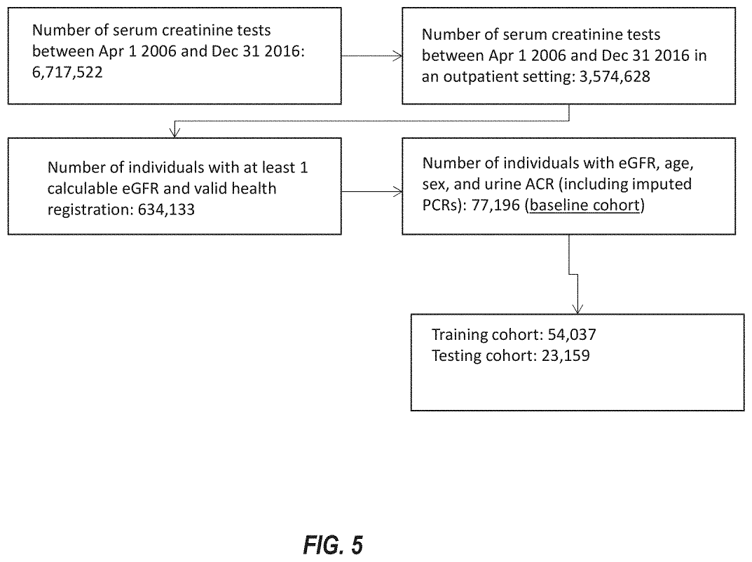

FIG. 5 schematically illustrates an example selection of a cohort of patients from which a machine learning model training dataset was generated. A study development cohort was derived from administrative data in Manitoba, Canada (at the time, population 1.4 million) using data from the Manitoba Centre for Health Policy (MCHP). The MCHP is a research unit within the Department of Community Health Sciences at the University of Manitoba that maintains a population-based repository of data on health services and other social determinants of health covering all individuals in the province. The training data set included all adult (age 18+) individuals in the province with an available outpatient eGFR test between Apr. 1, 2006, and Dec. 31, 2016, with valid Manitoba Health registration for at least 1 year pre-index. For example, eGFR was calculated from available serum creatinine tests using the CKD-EPI equation. Patients were further required to have demographic information on age and sex to be included, as well as the result of a urine albumin-to-creatinine ratio (ACR) or protein-to-creatinine ratio (PCR) test. Patients with a history of kidney failure (dialysis or transplant) were excluded. Data was de-identified using a scrambled personal health information number.

In the example study, the system identified 6,717,522 serum creatinine tests between Apr. 1, 2006 and Dec. 31, 2016, of which 3,574,628 were performed in an outpatient setting. From this, the system was able to identify 634,133 unique individuals with at least 1 calculable eGFR measurement and valid health registration. After restricting to the requirement of a valid urine ACR test (or converted PCR test) the system arrived at a total cohort size of 77,196 for both the training and testing datasets (FIG. 5). For evaluation of the outcome at 2 years, the training dataset included complete follow up in 61,353 individuals (42,947 in training and 18,406 in testing), and 35,736 individuals for evaluation of the outcome at 5 years (54,037 in training and 23,159 in testing).

In one example embodiment, the mean age of the baseline cohort was 59.3 years (±17.0), and patients had a mean eGFR of 82.2 (±27.2) ml/min/1.73 m². Median ACR after inclusion of converted PCRs was 1.1 mg/mmol (interquartile range 0.5 to 4.7 mg/mmol). 47.7% of patients were male, 45.2% had diabetes, and 69.9% had hypertension. 5.2%, 3.6%, and 2.6% had a history of congestive heart failure, stroke, or myocardial infarction, respectively. When split into training and testing groups, characteristics were similar.

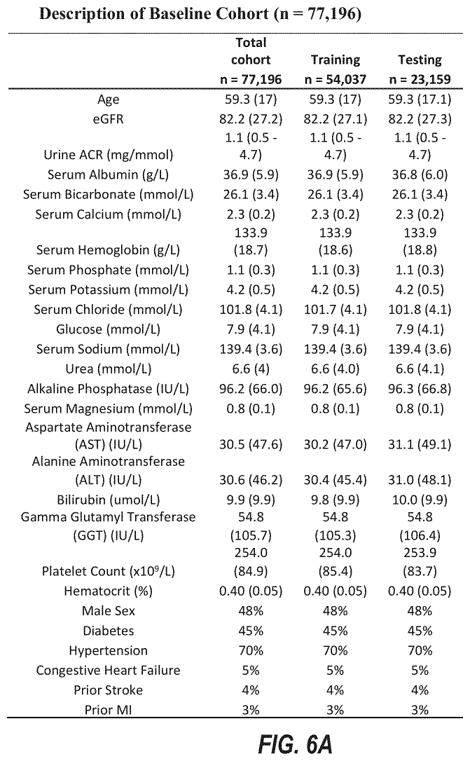

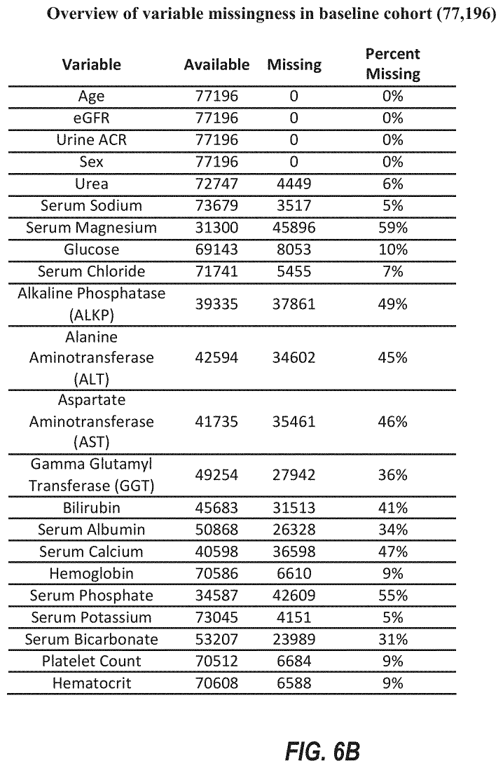

FIG. 6A illustrates a table comprising a description of the cohort discussed above with reference to FIG. 5, including various test results included in the medical laboratory data for each patient. The various test results were categorized as independent and dependent variables to be included in the training data set (e.g., training dataset 141).

Training datasets included age, sex, eGFR, and urine ACR as described above. Baseline eGFR was calculated as the average of all available eGFR results beginning with the first recorded eGFR during the study period and moving to the last available test in a 6-month window and calculating the mean of tests during this period. The index date of the patient was considered the date of the final eGFR in this 6-month period. Age was determined at the date of the index eGFR, and sex using a linkage to the Manitoba Health Insurance Registry which contains dates of birth and other demographic data. If a urine ACR test was unavailable, the available urine protein-to-creatinine (PCR) tests were converted to corresponding urine ACRs using published and validated equations. The closest result within 1 year of the index date was selected (before or after). Urine ACR was log-transformed due to the variables skewed distribution.

In addition to the previously described variables, other relevant laboratory variables were included that had a low degree of missingness in model creation (<15% or <30%). These included: serum sodium, serum chloride, serum hemoglobin, urea, serum potassium, glucose, AST, ALT, Bilirubin, GGT, Hematocrit, and/or platelet count. The closest value within 1 year of the index date is selected (before or after). The models constructed with these variables are referred to as “10 variable models” (age, sex, and the aforementioned labs).

When applied in cox proportional hazards models, multiple imputations (n=5) using SA PROC MI were applied. Random forest models allow for variables to be missing, with these observations having the “missing value” being treated as the splitting value of the variable in deciding branch splitting using SAS PROC HPFOREST. An additional random forest model is evaluated including 6 additional variables that allowed for any degree of missingness: serum albumin, alkaline phosphatase, serum phosphate, serum bicarbonate, serum magnesium, and serum calcium. This model is referred to as the 16-variable model. Laboratory data included in the training datasets is extractable from the Shared Health Diagnostic Services of Manitoba (DSM) Laboratory Information System.

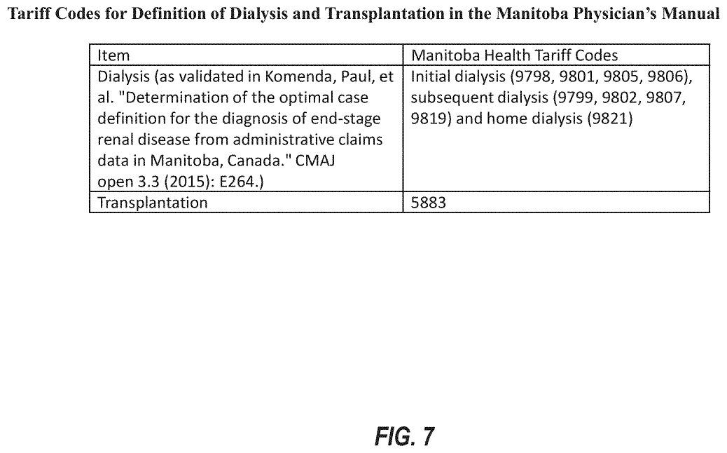

An outcome for at least some of the disclosed embodiments is prediction and/or risk score for a 40% decline in eGFR or kidney failure for a patient. Within the training dataset, the 40% decline in eGFR was determined as the first eGFR test that was 40% or greater in decline from the baseline eGFR, with a second confirmatory test at least 1 month after unless the patient died or experiences kidney failure in this 1-month period. The event date for the 40% decline is considered the first of these qualifying tests. Kidney failure was determined under three conditions: initiation of chronic dialysis, receipt of a transplant, or an eGFR <10 ml/min/1.73 m². Dialysis was defined as any 2 claims in the Manitoba Medical Services database for chronic dialysis, and transplant was defined as any 1 claim in the Manitoba Medical Services database for transplant or a hospitalization in the Discharge Abstract Database (DAD) with a corresponding procedure code for kidney transplantation (1PC85 or 1OK85 using the Canadian Classification of Health Interventions (CCI) codes). An overview of tariff codes identifying dialysis and transplant are provided in FIG. 7.

FIG. 6B is a table illustrating an overview of the degree of missingness of different variables in the baseline cohort. When applied in cox proportional hazards models, the system applied multiple imputations for variables with missingness <30% using SAS PROC MI. When applied in random forest models, the system applied imputations for missing data using a missing data algorithm. All laboratory data included was extracted from the Shared Health Diagnostic Services of Manitoba (DSM) Laboratory Information System and any values recorded during a hospitalization event as determined by a linkage to the Discharge Abstract Database (DAD) were not included.

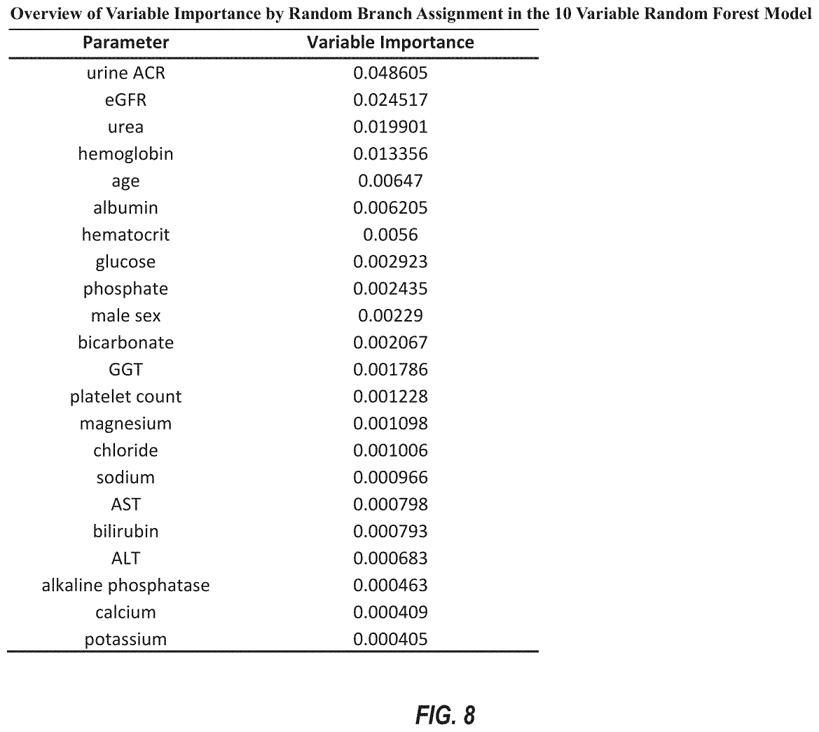

The outcome date for the 40% decline in eGFR or kidney failure was determined based on the first of these events. FIG. 8 is a table that illustrates an overview of variable importance for each variable included in a machine learning model training dataset. In particular, the table illustrates that for an example random forest model, the variables that had the highest impact in generating an accurate CKD progression prediction include the urine ACR, the eGFR, urea and hemoglobin. Age and sex are also meaningful variables.

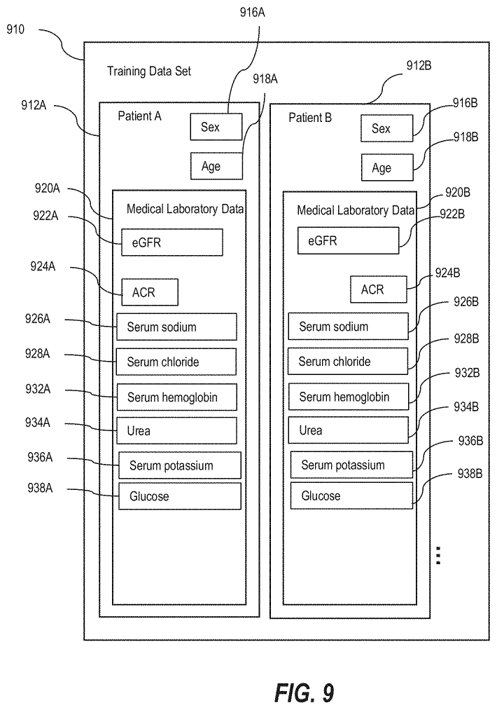

FIG. 9 conceptually depicts an example training dataset 910 that includes patient information (e.g., sex 916A, 916B, age 918A, 918B) and medical laboratory data for each patient included in the training dataset 910. As shown, the medical laboratory data 920A associated with patient A 912A includes a measurement for eGFR 922A, urine ACR 924A, serum sodium 926A, serum chloride 928A, serum hemoglobin 932A, urea 934A, serum potassium 936A, and glucose 938A. Similarly, as shown, the medical laboratory data 920B associated with patient B 912B includes a measurement for eGFR 922B, urine ACR 924B, serum sodium 926B, serum chloride 928B, serum hemoglobin 932B, urea 934B, serum potassium 936B, and glucose 938B. The ellipsis indicates that any number of patients may be included in the training dataset 910. As noted above, certain measurements may be missing for one or more patients represented in the training dataset 910.

Random forest models can be fit using the R package Fast Unified Random Forest for Survival, Regression, and Classification (RF-SRC) using a survival forest with right-censored survival. To accomplish this, data was split into training (70%) and testing (30%) datasets. Models were evaluated for accuracy using the time-dependent area under the receiver operating characteristic (ROC) curve, the Brier score, and a calibration plot of observed versus predicted risk. In addition, in this particular example, the system assessed sensitivity, specificity, negative predictive value (NPC), and positive predictive value (PPV) for the top 10%, 15%, and 20% of patients by estimated risk (high risk), as well as in the lowest 50%, 45%, and 30% of estimated risk (low risk).

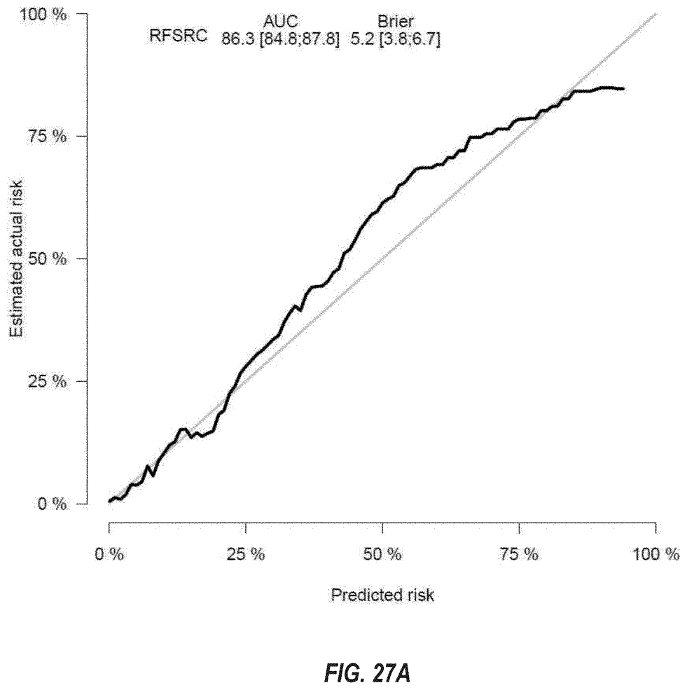

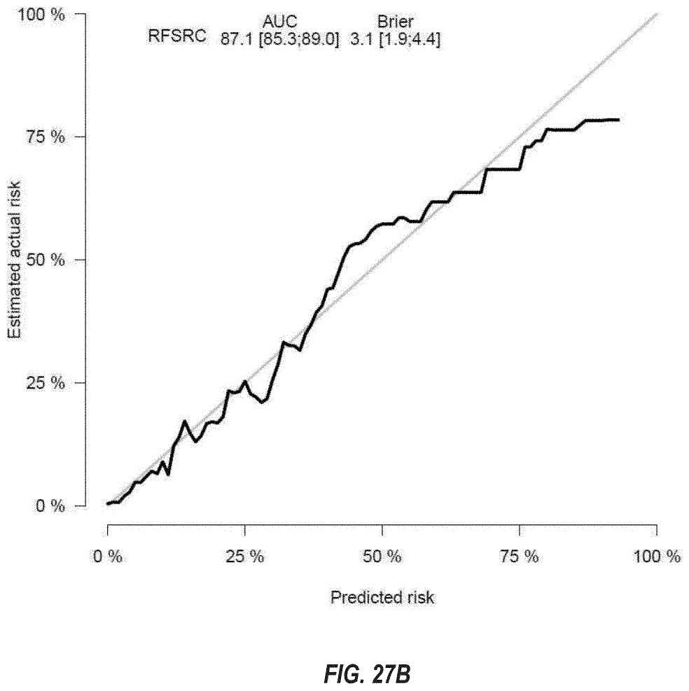

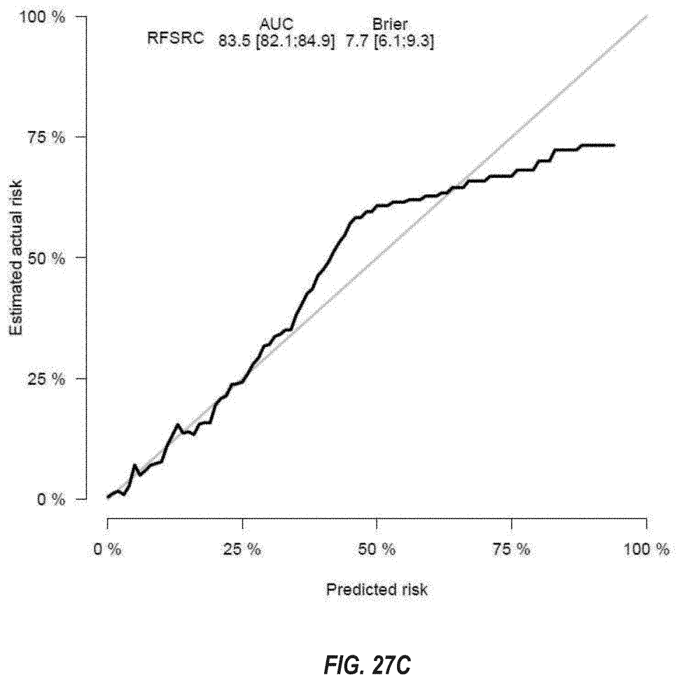

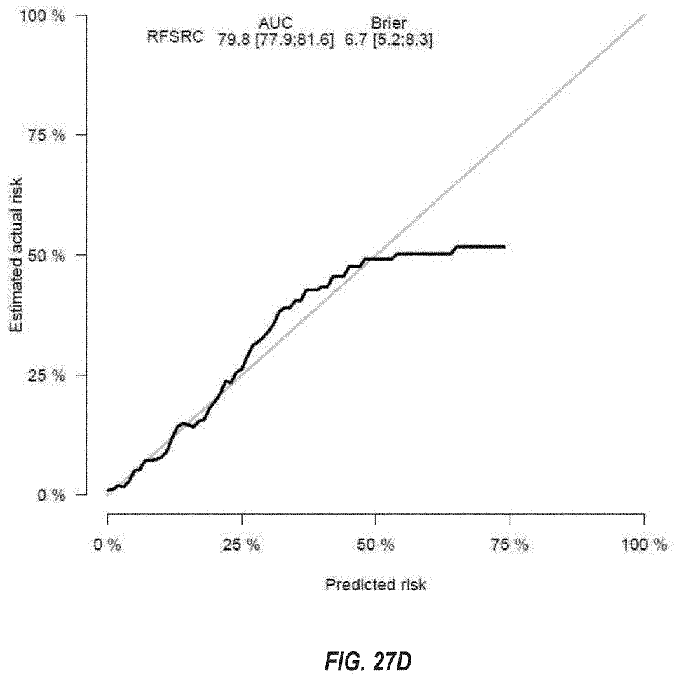

To evaluate generalizability, the system evaluated the model in subpopulations of the testing cohort, including: (1) patients with diabetes; (2) patients without diabetes; (3) patients with CKD as defined by eGFR<60 ml/min/1.73 m² or urine ACR>3 mg/mmol (including converted urine PCR tests); and (4) patients with CKD stages G1-G3 as defined by patients with eGFR 30-60 ml/min/1.73 m² or eGFR>60 ml/min/1.73 m² and urine ACR>3 mg/mmol (including converted urine PCR tests). See FIGS. 27A-27B. Using the final grown 22 variable forest, variable importance of included parameters was evaluated.

Cox proportional hazard models were also developed in the training dataset: (1) a model with variables that had at most 30% missingness (11 variable model); and (2) a model with the variables age, sex, eGFR, and urine ACR to compare with the Kidney Failure Risk Equation (KFRE). Model discrimination was assessed using Harrell's c-statistic, accuracy using the Brier score, and calibration using a plot of observed versus predicted risk probabilities in the testing dataset. Analysis was performed using SAS Version 9.4 (Cary, N.C.) and R Version 4.1.0. Statistical significance was a priori identified using an alpha=0.05.

Random forest models were also fit using SAS PROC HPFOREST and internally validated using SAS PROC HP4SCORE using the various demographic and laboratory variables. In some statistical analysis results, the out of bag (OOB) misclassification rate was examined against the number of leaves selected in the model. Measures of accuracy for prediction of the outcome at 2 and 5 years were evaluated for the random forest model, including the area under the receiving operating characteristic (ROC) curve, the Brier score, a calibration plot of observed and predicted risks by risk decile of predicted probabilities.

In addition, other parameters were assessed including sensitivity, specificity, negative predictive value (NPV), and positive predictive value (PPV) at cut-offs of 1% and 10% in the 2-year model, and 5% and 25% in the 5-year model. These cut-offs were selected as they were clinically meaningful and correspond to approximately the bottom 60% and top 10% of individuals as classified by predicted risk scores. A measurement of variable importance using the random branch assignments (RBA) method in SAS PROC HP4SCORE was computed to evaluate the square error loss.

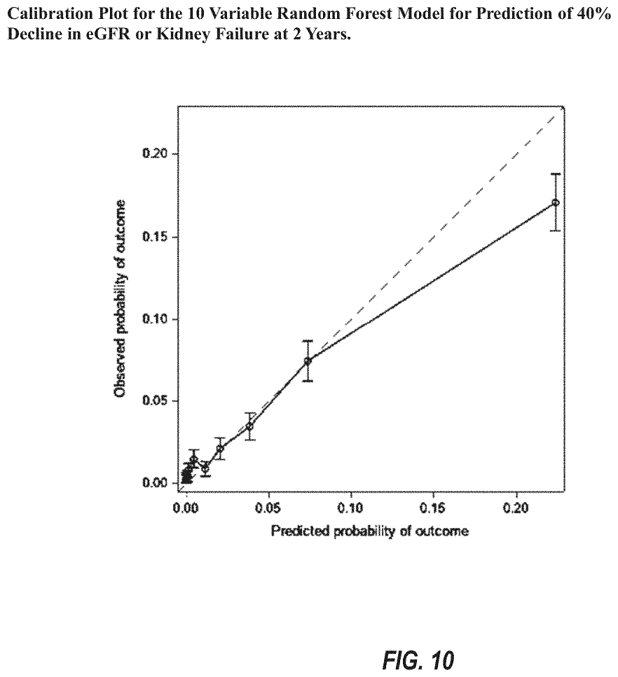

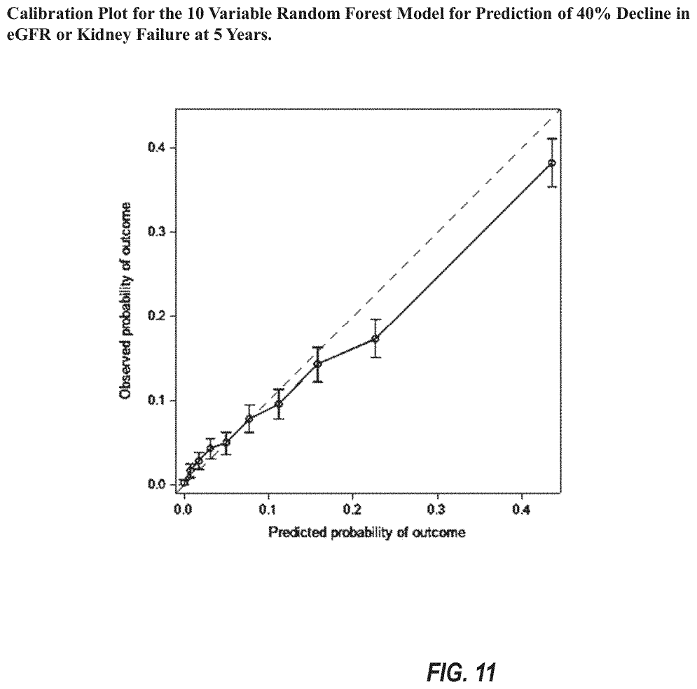

For example, FIG. 10 is a graph illustrating an example calibration plot for a machine learning model configured as a random forest model, for example, using the training dataset shown in FIG. 9, for predicting decline within a time period of two years. FIG. 11 is a graph illustrating an example calibration plot for a machine learning model configured as a random forest model, for example, using the training dataset as shown in FIG. 9, for a time period of five years. For the example implemented, as is evident from the graphs depicted in FIGS. 10-11, 5-year prediction (FIG. 11) was correlated more closely with observed outcomes than 2-year prediction (FIG. 10), but both predictive models provided useful predictive metrics that can guide patient care and/or treatment/prevention decisions.

The study also analyzed various developed Cox proportional hazard models in the training dataset with the above variables to predict the risk of developing the outcome of a 40% decline or kidney failure, and subsequently internally validated them in the testing set. Model discrimination was assessed at 2- and 5-years using Harrell's c-statistic, accuracy using the Brier score, and calibration using a plot of observed versus predicted risk probabilities by decile of predicted risk. All analysis was performed using SAS Version 9.4 (Cary, N.C.). Statistical significance was a priori identified using an alpha=0.05.

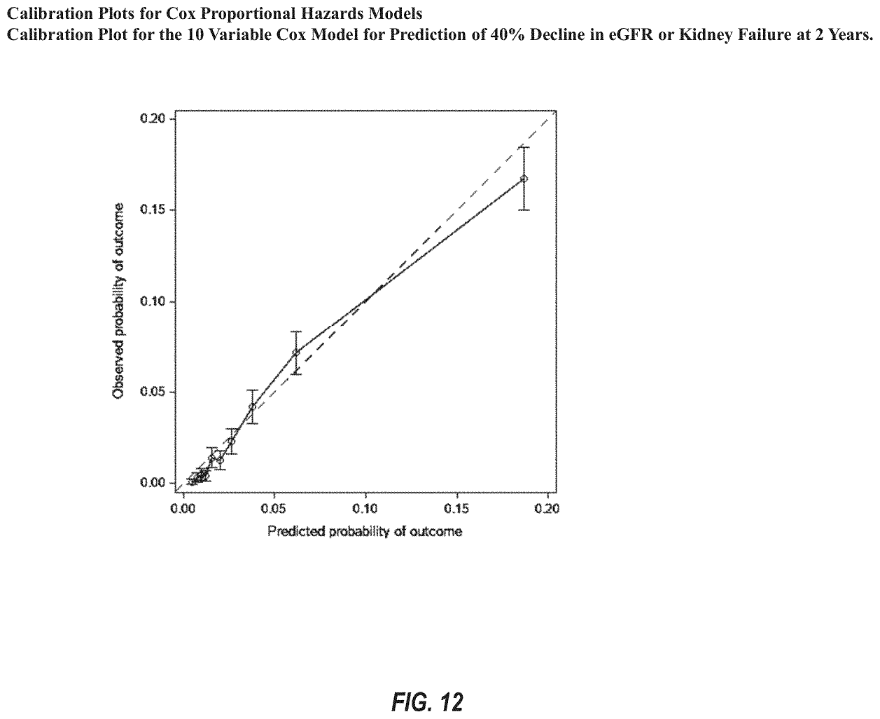

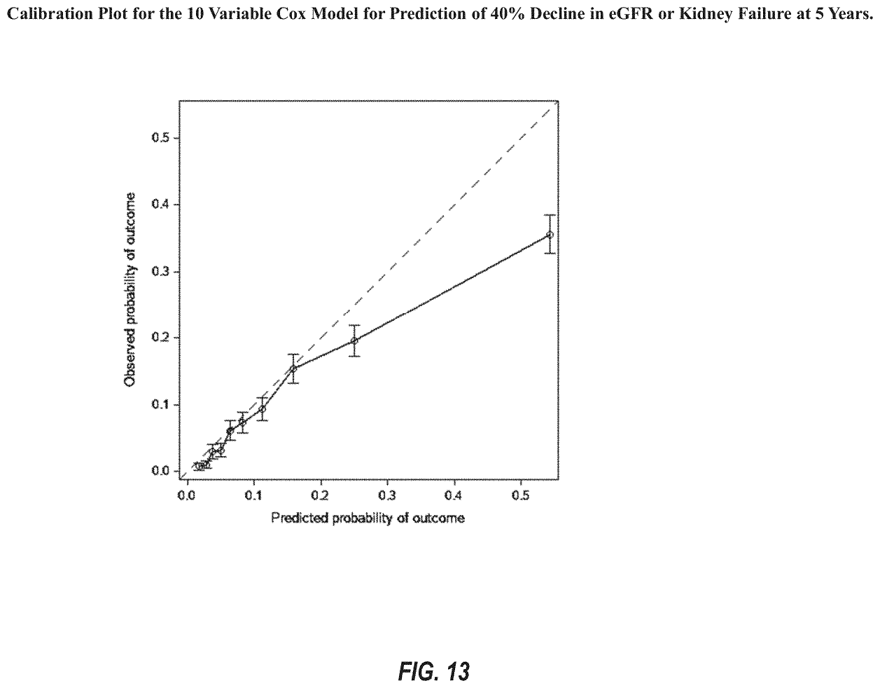

For example, FIG. 12 is a graph illustrating an example calibration plot for a machine learning model configured as a Cox model, for example, using the training dataset as shown in FIG. 9, for a time period of two years. FIG. 13 is a graph illustrating an example calibration plot for a machine learning model configured as a Cox model, for example, using the training dataset shown in FIG. 9, for a time period of five years. For the example implemented, as is evident from the graphs depicted in FIGS. 12-13, 2-year prediction (FIG. 12) correlated more closely with observed outcomes than 5-year prediction (FIG. 13), but both predictive models provided useful predictive metrics that can guide patient care and/or treatment/prevention decisions. Furthermore, it is observed that the 10 variable Cox model provided better correlation to observed outcomes at 2 years (FIG. 12) when compared to the 10 variable Random Forest Model (FIG. 10).

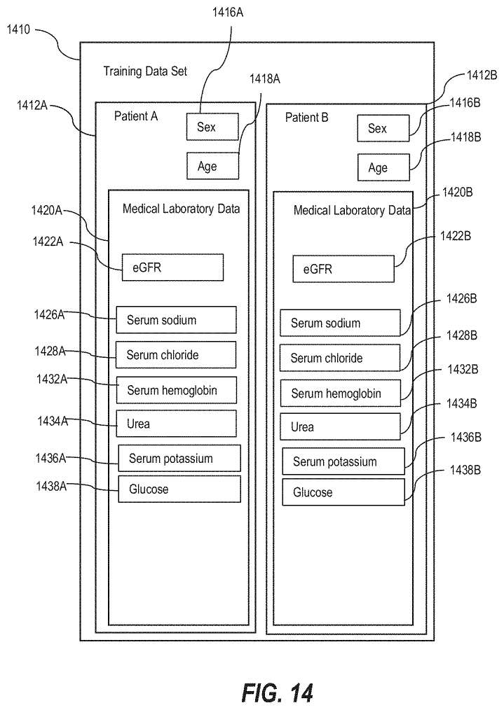

FIG. 14 conceptually depicts an example training dataset 1410 that includes patient information (e.g., sex 1416A, 1416B, age 1418A, 1418B) and medical laboratory data for each patient included in the training dataset 1410, usable to form a 9 variable model for predicting CKD progression. The training dataset 1410 is similar to the training dataset 910 of FIG. 9, while omitting urine ACR measurements. As shown, the medical laboratory data 1420A associated with patient A 1412A includes a measurement for eGFR 1422A, serum sodium 1426A, serum chloride 1428A, serum hemoglobin 1432A, urea 1434A, serum potassium 1436A, and glucose 1438A. Similarly, as shown, the medical laboratory data 1420B associated with patient B 1412B includes a measurement for eGFR 1422B, serum sodium 1426B, serum chloride 1428B, serum hemoglobin 1432B, urea 1434B, serum potassium 1436B, and glucose 1438B. Any number of patients may be included in the training dataset 1410. As noted above, certain measurements may be missing for one or more patients represented in the training dataset 1410.

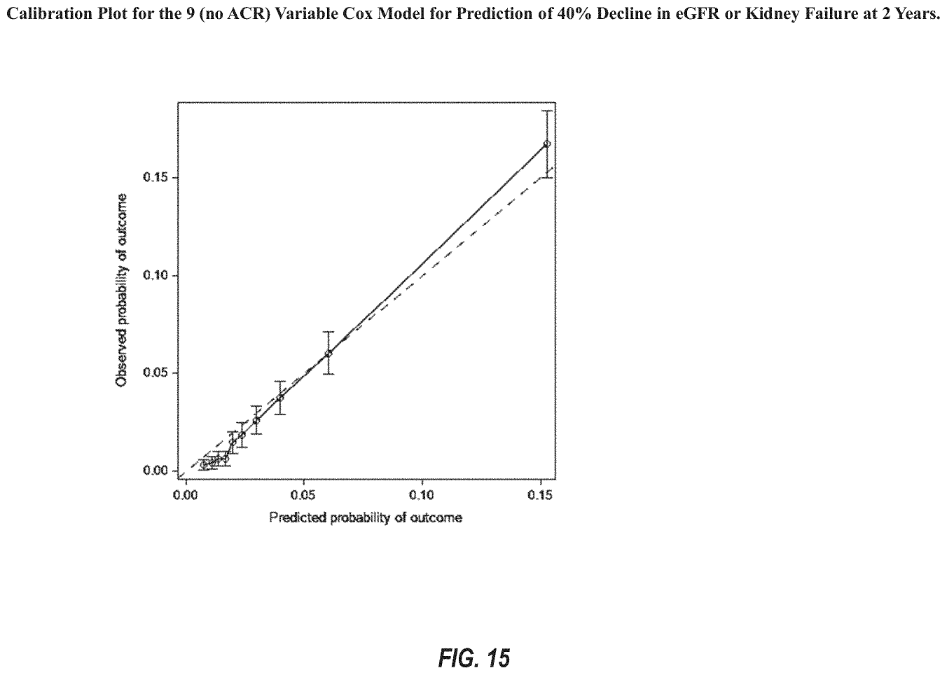

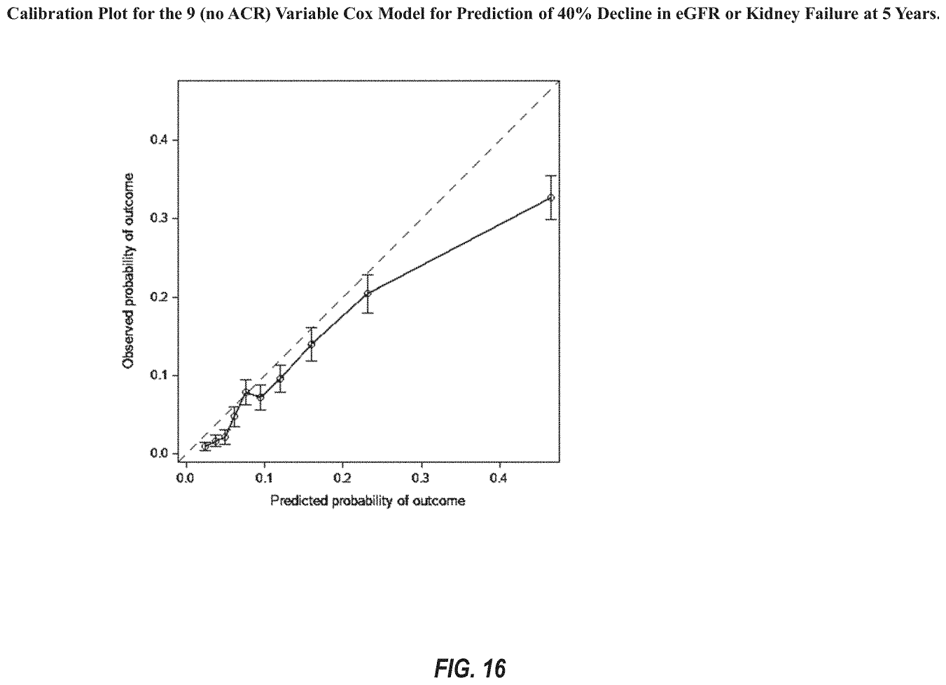

FIG. 15 is a graph illustrating an example calibration plot for a machine learning model configured as a Cox model, for example, using the training dataset as shown in FIG. 14, for a time period of two years. FIG. 16 is a graph illustrating an example calibration plot for a machine learning model configured as a Cox model, for example, using the training dataset as shown in FIG. 14, for a time period of five years. For the example implemented, as is evident from the graphs depicted in FIGS. 15 and 16, 2-year prediction (FIG. 15) correlated more closely with observed outcomes than 5-year prediction (FIG. 16), but both predictive models provided useful predictive metrics that can guide patient care and/or treatment/prevention decisions. It should also be noted that the 2-year prediction and 5-year prediction using the 9-variable model (FIGS. 15 and 16) produced similar correlation results to the 2-year prediction and 5-year prediction using the 10-variable model (FIGS. 12 and 13), showing that omitting the ACR can still provide closely correlated predictive power for either timeframe.

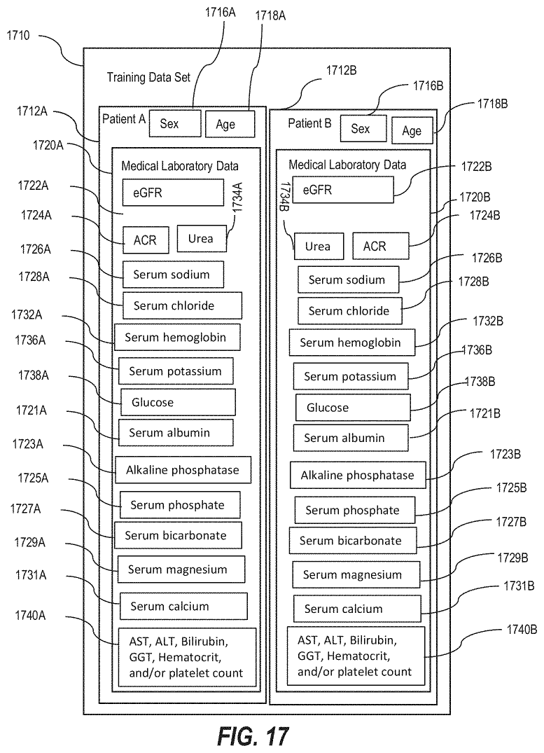

FIG. 17 illustrates an example training dataset 1710 comprising a 16 to 22 variable medical laboratory dataset, which can be used to train a machine learning model configured to generate a prediction of chronic kidney disease progression. Training dataset 1710 is example of training dataset 910 in FIG. 9 (including sex 1716A and 1716B and age 1718A and 1718B for patient A 1712A and patient B 1712B, respectively), with additional measurements included in the medical laboratory data for at least some patients included in the training dataset 1710.

As shown, the medical laboratory data 1720A associated with patient A 1712A includes a measurement for eGFR 1722A, urine ACR 1724A, serum sodium 1726A, serum chloride 1728A, serum hemoglobin 1732A, urea 1734A, serum potassium 1736A, glucose 1738A, serum albumin 1721A, alkaline phosphatase 1723A, serum phosphate 1725A, serum bicarbonate 1727A, serum magnesium 1729A, and serum calcium 1731A.

Similarly, as shown, the medical laboratory data 1720B associated with patient B 1712B includes a measurement for eGFR 1722B, urine ACR 1724B, serum sodium 1726B, serum chloride 1728B, serum hemoglobin 1732B, urea 1734B, serum potassium 1736B, glucose 1738B, serum albumin 1721B, alkaline phosphatase 1723B, serum phosphate 1725B, serum bicarbonate 1727B, serum magnesium 1729B, and serum calcium 1731B. In some embodiments, the medical laboratory data 1720A of patient A and the medical laboratory data 1720B of patient B further include AST, ALT, bilirubin, GGT, hematocrit and/or a platelet count 1740A and 1740B, respectively. Any number of patients may be included in the training dataset 1710. As noted above, certain measurements may be missing for one or more patients represented in the training dataset 1710.

In some embodiments, a machine learning model trained using training dataset 1710 is configured as a 22 variable model. Thus, the input data set of the new patient may also include as many as the 22 different laboratory data points/measurements (or possibly more).

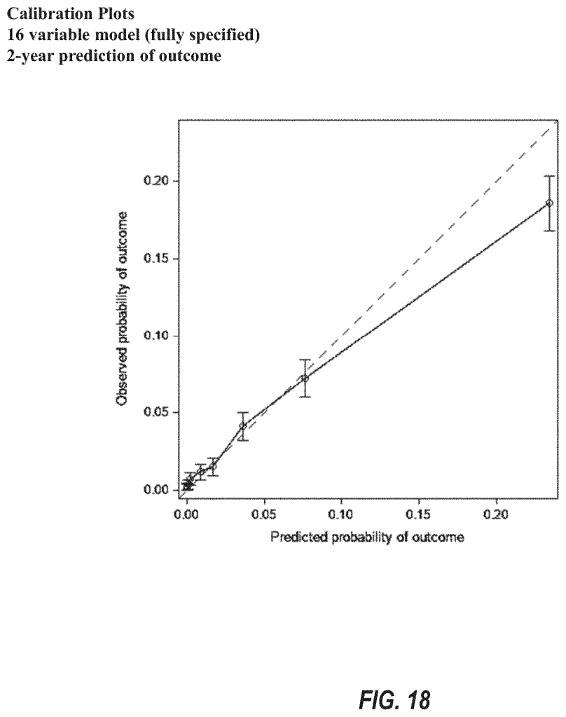

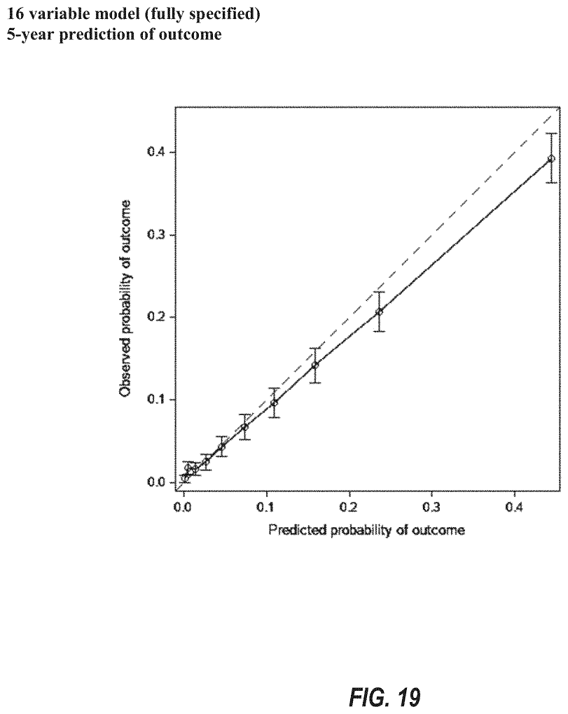

FIG. 18 is a graph illustrating an example calibration plot for a machine learning model, for example, using 16 variables of the training dataset as shown in FIG. 17, for a time period of two years. FIG. 19 is a graph illustrating an example calibration plot for a machine learning model, for example, using 16 variables of the training dataset as shown in FIG. 17, for a time period of five years. For the example implemented, as is evident from the graphs depicted in FIGS. 18 and 19, 5-year prediction (FIG. 19) correlated more closely with observed outcomes than 2-year prediction (FIG. 18), but both predictive models provided useful predictive metrics that can guide patient care and/or treatment/prevention decisions. Furthermore, it should be noted that for the 2-year predictions, the 16-variable model (FIG. 18) showed an improvement in correlation when compared to the 10-variable model (FIG. 10). However, for the 5-year prediction, both the 16-variable model (FIG. 19) and the 10-variable model (FIG. 11) both performed substantially equivalently for the 40% prediction threshold. The 16-variable model (FIG. 19) provided a more stable correlation through the lower percentage thresholds than the 10-variable model (FIG. 11).

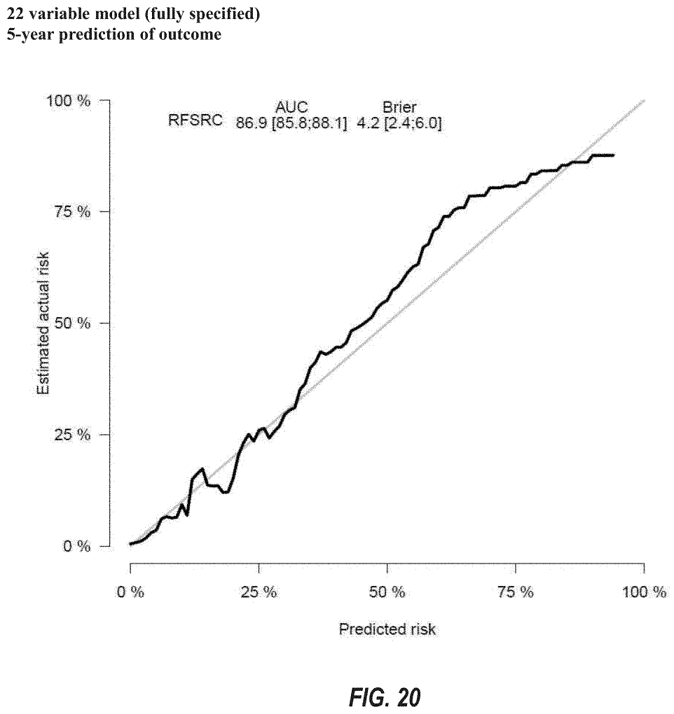

FIG. 20 is a graph illustrating a calibration plot for a 22 variable random forest model for prediction of a 40% decline in eGFR or Kidney Failure at 5 years.

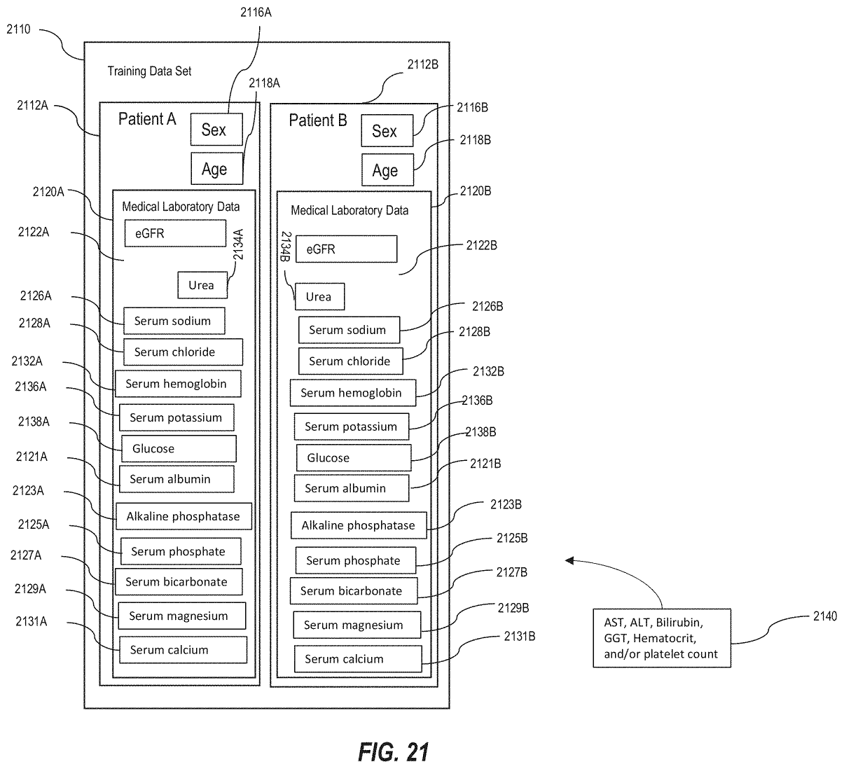

FIG. 21 illustrates an example training dataset 2110 comprising a 15 to 21 variable medical laboratory dataset, which can be used to train a machine learning model configured to generate a prediction of chronic kidney disease progression. The training dataset 2110 is example of training dataset 1710 of FIG. 17 (including sex 2116A and 2116B and age 2118A and 2118B for patient A 2112A and patient B 2112B, respectively), with the exception of excluding the measurement of urine ACR for each patient included in the training dataset 2110.

As shown, the medical laboratory data 2120A associated with patient A 2112A includes a measurement for eGFR 2122A, serum sodium 2126A, serum chloride 2128A, serum hemoglobin 2132A, urea 2134A, serum potassium 2136A, glucose 2138A, serum albumin 2121A, alkaline phosphatase 2123A, serum phosphate 2125A, serum bicarbonate 2127A, serum magnesium 2129A, and serum calcium 2131A.

Similarly, as shown, the medical laboratory data 2120B associated with patient B 2112B includes a measurement for eGFR 2122B, serum sodium 2126B, serum chloride 2128B, serum hemoglobin 2132B, urea 2134B, serum potassium 2136B, glucose 2138B, serum albumin 2121B, alkaline phosphatase 2123B, serum phosphate 2125B, serum bicarbonate 2127B, serum magnesium 2129B, and serum calcium 2131B. In some embodiments, the medical laboratory data 2120A of patient A and the medical laboratory data 2120B of patient B further include AST, ALT, bilirubin, GGT, hematocrit and/or a platelet count 2140. Any number of patients may be included in the training dataset 2110. As noted above, certain measurements may be missing for one or more patients represented in the training dataset 2110.

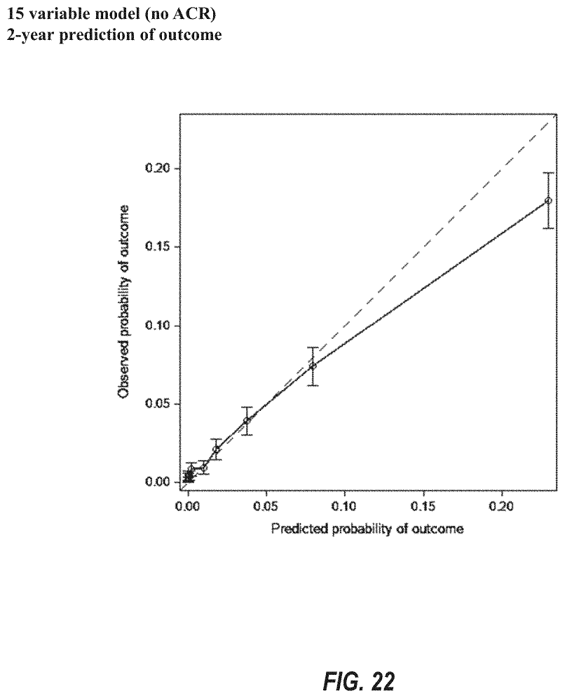

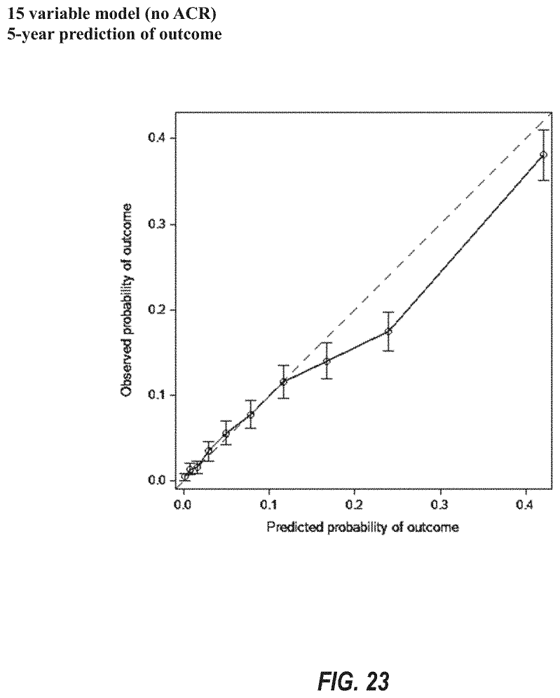

FIG. 22 is a graph illustrating an example calibration plot for a machine learning model, for example, using the training dataset (15-variable) as shown in FIG. 21, for a time period of two years. FIG. 23 is a graph illustrating an example calibration plot for a machine learning model, for example, using the training dataset (15-variable) as shown in FIG. 21, for a time period of five years. In the example implemented, as shown in the graphs depicted in FIGS. 22 and 23, 5-year prediction (FIG. 23) correlated more closely with observed outcomes than 2-year prediction (FIG. 22), but both predictive models provided useful predictive metrics that can guide patient care and/or treatment/prevention decisions. Furthermore, the 15-variable model (FIG. 23) performed similarly to the 16-variable model (FIG. 19) for 5-year prediction, suggesting that the omission of ACR did not significantly affect the predictions provided by the models.

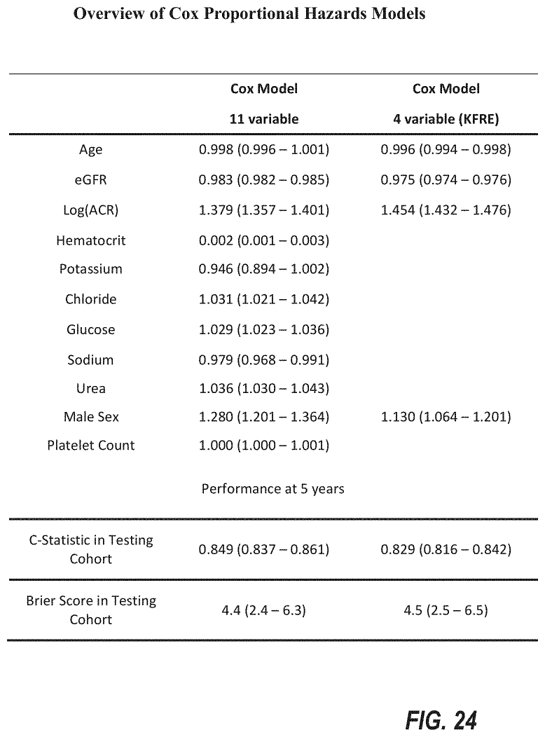

FIG. 24 is a table illustrating an example overview of performance evaluation statistics for various example machine learning models with 4 to 11 variables as disclosed herein and configured as Cox models. As illustrated in FIG. 24, various models were evaluated against a predicted performance at 5 years. Variables that were considered include age, eGFR, log transformed ACR, Hematocrit, Potassium, Chloride, Glucose, Sodium, Urea, Male Sex, and a platelet count.

In other tests (not illustrated), the system evaluated the Cox proportional hazards models in cohorts that had fully available follow up at 2 and 5 years to compare them to the output of the Random Forest models below. For the prediction of the outcome at 2 years in the testing cohort, the Cox proportional hazards model had a c-statistic of 0.8492 (SE 0.007) in the baseline model, decreasing to 0.8151 (0.006) at 5 years.

In the models where urine ACR was removed (e.g., the 9 and 15 variable models), the system found a c-statistic of 0.8266 (0.008) at 2 years and 0.7942 (0.006) at 5 years. In the model applying the cohort with 2 years of follow up, the Brier score was 0.0298 (0.001) for the prediction of the eGFR decline or kidney failure outcome, and for the cohort with 5 years of follow up the Brier score was 0.0832 (0.002) in the testing cohort. In the models where urine ACR was removed, the Brier score was 0.0305 (0.001) for the prediction of the outcome at 2 years, and 0.0855 (0.002) for the prediction of the outcome at 5 years.

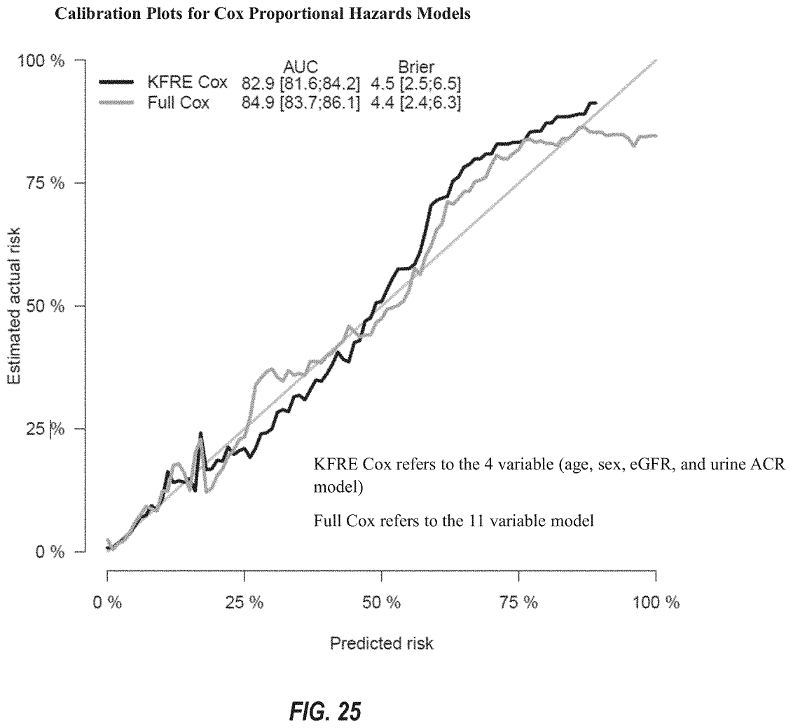

FIG. 25 is a graph illustrating a calibration plot for cox proportional hazard models, including a 4 variable model and an 11 variable model. Both models performed well, with accurately predicting risk. The different Cox proportional hazards models were evaluated with a maximum follow up time of 5 years for the outcome of 40% decline in eGFR or kidney failure, censoring for death and loss to follow up. These included: (1) an 11 variable model including all variables that had 30% missingness or less: age, eGFR, male sex, urine ACR, platelet count, potassium, hematocrit, serum chloride, glucose, serum sodium, and urea; and (2) a 4-variable model with age, eGFR, male sex, and urine ACR. The 11 variable Cox model had a Harrell's c statistic of 0.849 (95% confidence interval of 0.837 to 0.861) and a Brier score of 4.4 (2.4 to 6.3) and was well calibrated at all levels of risk. Similarly, the 4 variable Cox model had a Harrell's c statistic of 0.829 (0.816 to 0.842) and a Brier score of 4.5 (2.5-6.5) and had similar calibration, as shown in FIG. 25.

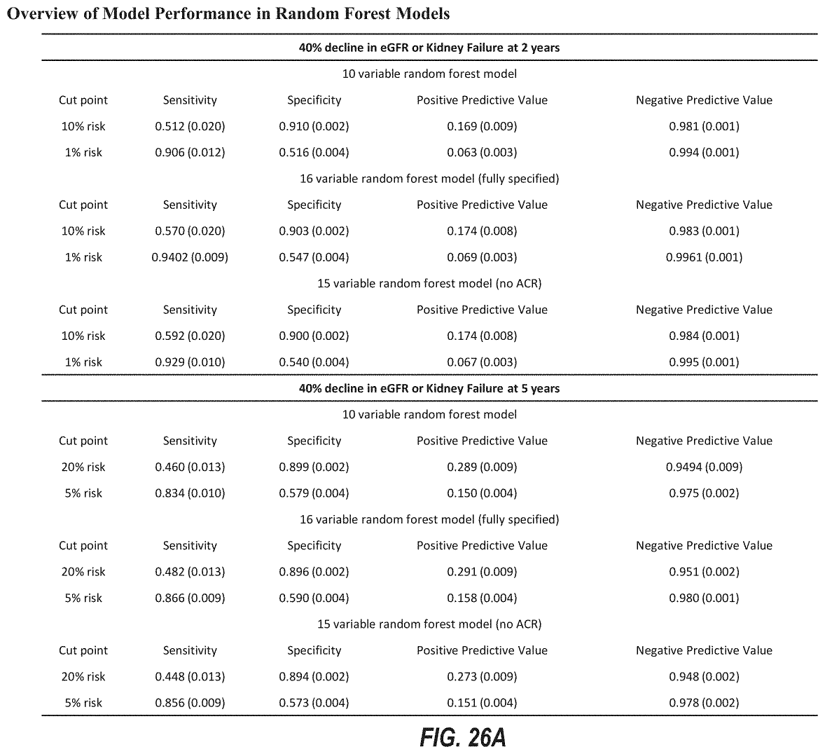

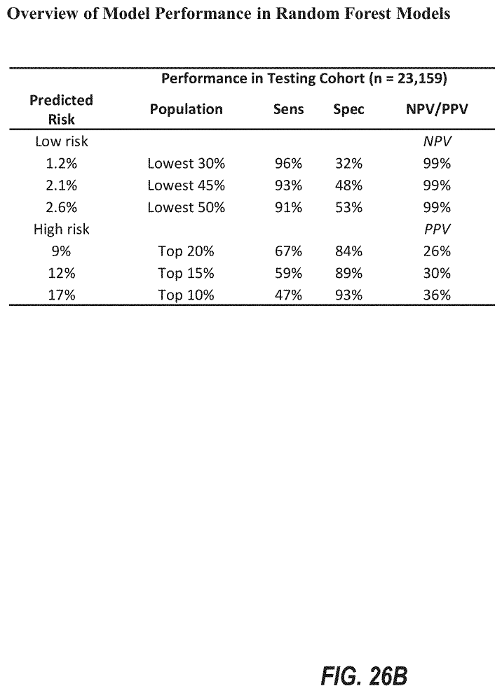

FIG. 26A is a table illustrating an example overview of performance evaluation statistics for various example machine learning models configured as random forest models. In the random forest model with 10 variables, the system found excellent discrimination with an area under the ROC of 0.8406 (SE 0.0080) at 2 years, and 0.7966 (0.0069) at 5 years. With respect to accuracy, the system found a Brier score at 2 years of 0.029 (SE 0.001), and at 5 years of 0.077 (0.002). In the baseline model at 2 and 5 years, the system observed excellent calibration. In the 16-variable random forest, c-statistics were 0.8697 (0.007) for the prediction of the outcome at 2 years and 0.8190 (0.006) at 5 years. When excluding ACR from this model the c-statistic at 2 years was 0.8597 (0.007) and was 0.8014 (0.007) at 5 years. Additional model metrics and calibration plots for the 16 variable and 15 variable (excluding ACR) models are provided in the corresponding figures.

FIG. 26B is another table illustrating the overview of model performance in random forest models (the 22 variable version of the machine learning model described above). Low risks were determined to be between 1.2% and 2.6%. High risks were determined to be between 9% and 17%. The performance was evaluated in a testing cohort of 23,159 patients. In the random forest model with 22 variables, the system also found excellent discrimination with a time dependent area under the receiver operating characteristic (AUROC) curve of 86.9 (95% CI 85.8 to 88.1) over the maximum 5 year follow up, and a Brier score of 4.2 (2.5 to 6.0). The results observed included excellent calibration. Similar performance was observed in all subgroups: diabetes (AUROC: 86.3; Brier: 5.2), without diabetes (AUROC: 87.1; Brier: 3.1), CKD (AUROC: 83.5; Brier: 7.7), CKD stages G1-G3 (AUROC: 79.8, Brier: 6.7).

Statistics on sensitivity, specificity, and positive predictive value were evaluated in high-risk patients (top 10, 15, and 20% of risk scores respectively). The evaluation tests found that sensitivity was 47% in the top 10% of risk scores (17% 5-year risk threshold), with a specificity of 93% and positive predictive value of 36%. In the top 15% (12% 5-year risk threshold), sensitivity was 59%, specificity 89%, and positive predictive value 30%. In the top 20% (9% 5-year risk threshold), the model had a sensitivity of 67%, specificity of 84%, and positive predictive value of 26%).

Likewise, the system evaluated sensitivity, specificity, and negative predictive value in low-risk patients (bottom 50, 45, and 30% of patients respectively). In the lowest 50% of patients (2.6% 5-year risk threshold), the model had a sensitivity of 91%, specificity of 53%, and negative predictive value of 99%. For the lowest 45% of patients (2.1% 5-year risk threshold), the model had a sensitivity of 93%, specificity of 48%, and negative predictive value of 99%. Lastly, in the lowest 30% of patients (1.2% 5-year risk threshold), the model had a sensitivity of 96%, specificity of 32%, and negative predictive value of 99%.

FIGS. 27A-27D illustrate various calibration plots for a 22 variable model configured as a random forest model is various subgroups. For example, FIG. 27A shows a calibration plot for the subgroup of patients with diabetes. FIG. 27B shows a calibration plot for the subgroup of patients without diabetes. FIG. 27C shows a calibration plot for patients with eGFR<60 ml/min/1.73 m² or urine ACR>3 mg/mmol, including converted urine PCRs. FIG. 27D illustrates a calibration plot for a subgroup of patients with CKD stages G1-G3 (e.g., eGFR is between 30-60 ml/min/1.73 m\hat{ }2 or eGFR>60 ml/min/1.73 m\hat{ }2 and urine ACR>3 mg/mmol, including converted urine PCRs.

### Random Survival Forest Model Example(s)

To develop one example random survival forest model for generating predictions of CKD progression, the development cohort was derived from administrative data in Manitoba, Canada (population 1.4 million), using data from the Manitoba Centre for Health Policy. All adult (age 18+ years) individuals in the province with an available outpatient eGFR test between Apr. 1, 2006, and Dec. 31, 2016, with valid Manitoba Health registration for at least 1-year pre-index were identified. eGFR was calculated from available serum creatinine tests using the CKD-Epidemiology Collaboration equation. Included patients were further required to have complete demographic information on age and sex, including the result of at least 1 urine ACR or protein-to-creatinine ratio (PCR) test. Patients with a history of kidney failure (dialysis or transplant) were excluded. The cohort discussed above with reference to FIG. 5 was used to develop the random survival forest model.

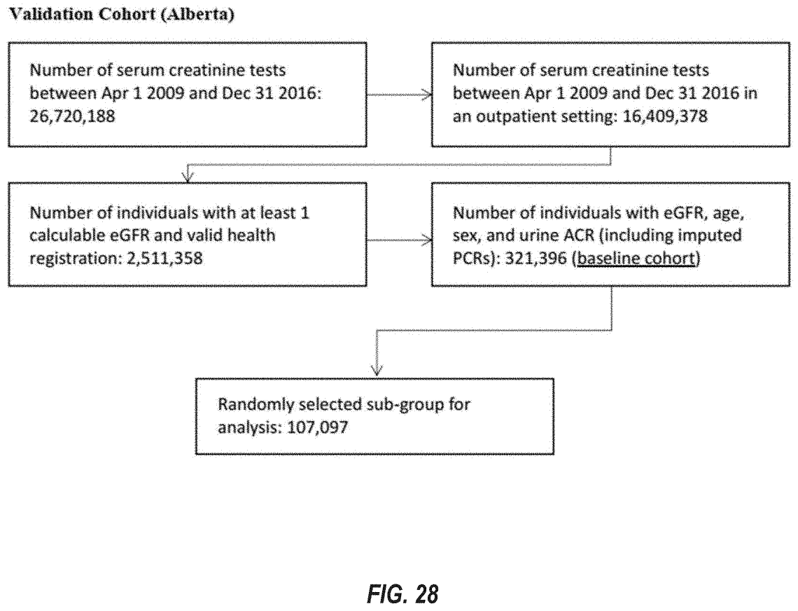

The validation cohort was derived from the Alberta Health database. This database contains information on demographic data, laboratory data, hospitalizations, and physician claims for all patients in the province of Alberta, Canada (population 4.4 million). Regular laboratory coverage for creatinine measurements and ACR/PCR values is complete from 2005; however, additional laboratory values are fully covered only from 2009 onward. As such, a cohort of individuals with at least 1 calculable eGFR, valid health registration, and an ACR (or imputed PCRs) value starting from Apr. 1, 2009, to Dec. 31, 2016 were identified. One-third of the external cohort were randomly sampled to perform the final analysis to reduce computation time. Patients with a history of kidney failure (dialysis or transplant) were excluded. FIG. 28 illustrates aspects of the validation cohort used to externally validate the random survival forest model.

To develop the random survival forest model, all candidate models included age, sex, eGFR, and urine ACR (e.g., as described previously). Baseline eGFR was calculated as the average of all available outpatient eGFR results beginning with the first recorded eGFR during the study period and moving forward to the last available test in a 6-month window and calculating the mean of tests during this period. The index date of the patient was considered the date of the final eGFR in this 6-month period. Age was determined as the date of the index eGFR, and sex was determined using a linkage to the Manitoba Health Insurance Registry which contained dates of birth and other demographic data. If a urine ACR test was unavailable, available urine PCR tests were converted to corresponding urine ACRs using published and validated equations. The closest result within 1 year before or after the index date was selected. Urine ACR was log transformed to handle the skewed distribution.

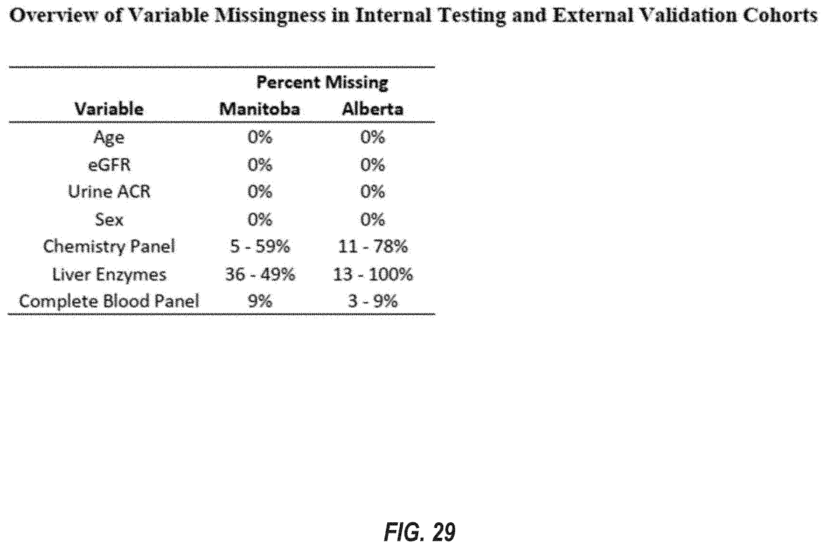

In addition to the previously described variables (age, sex, eGFR, and urine ACR), the utility of additional laboratory results from chemistry panels, liver enzymes, and complete blood cell count panels were evaluated for inclusion in the random forest model for survival. The closest value within 1 year of the index date was selected for inclusion. Distributional transformations were applied when needed. The final random survival forest model included eGFR, urine ACR, and an additional 18 laboratory results (i.e., urea, serum sodium, serum chloride, serum hemoglobin, serum potassium, glucose, serum albumin, alkaline phosphatase, serum phosphate, serum bicarbonate, serum magnesium, serum calcium, AST, ALT, bilirubin, GGT, hematocrit, and platelet count). An overview of the degree of missingness for the laboratory panels is provided in FIG. 29. The random forest models applied imputations for missing data using the method of adaptive tree imputation.

All laboratory data included were extracted from the Shared Health Diagnostic Services of Manitoba Laboratory Information System, and any values recorded during a hospitalization event as determined by a linkage to the Discharge Abstract Database were not included (inpatient tests). For the validation cohort, Alberta Health laboratory data were extracted from the Alberta Kidney Disease Network. Of the 18 laboratory tests used in the Manitoba model, 16 were also regularly collected by the Alberta Kidney Disease Network. The unavailable tests (aspartate aminotransferase and gamma glutamyl transferase) were treated as missing data.

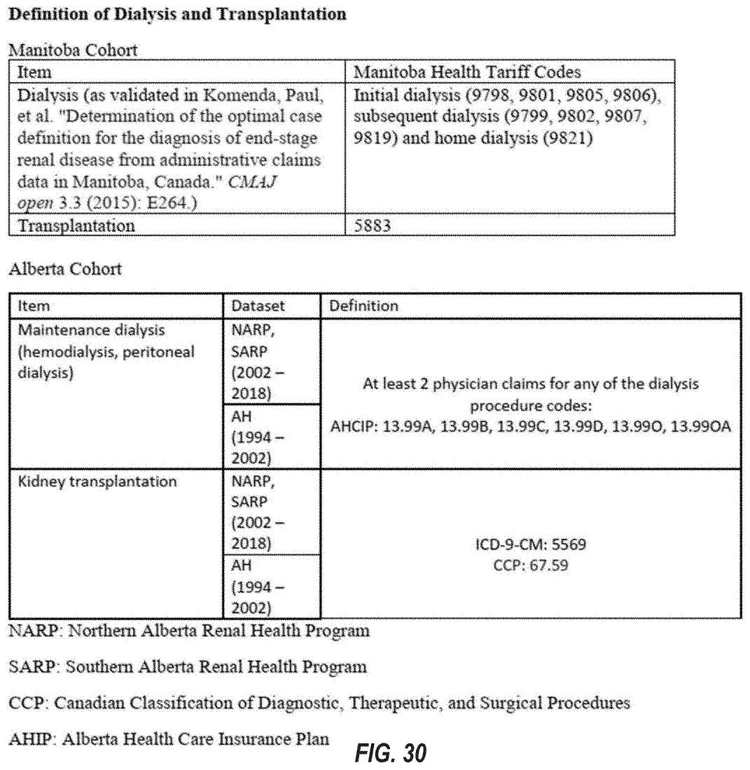

The primary outcome in the present example was a 40% decline in eGFR or kidney failure. The 40% decline in eGFR was determined as the first eGFR test in the laboratory data that was 40% or greater in decline from the baseline eGFR, requiring a second confirmatory test result between 90 days and 2 years after the first test unless the patient dies or experiences kidney failure within 90 days after the first test result revealing a 40% or greater decline. Therefore, a patient experiencing a single eGFR representing a 40% decline and dying within 90 days is treated as an event, or if they experience kidney failure in that period. Kidney failure was defined as initiation of chronic dialysis, receipt of a transplant, or an eGFR <10 ml/min per 1.73 m². Dialysis was defined as any 2 claims in the Manitoba Medical Services database for chronic dialysis, and transplant was defined as any 1 claim in the Manitoba Medical Services database for kidney transplant or a hospitalization in the Discharge Abstract Database with a corresponding procedure code for kidney transplantation (1PC85 or 1OK85 using the Canadian Classification of Health Interventions codes or International Classification of Diseases, Ninth Revision, procedure code 55.6). An overview of tariff codes identifying dialysis and transplant is provided in FIG. 30.

The outcome date for the 40% decline in eGFR or kidney failure was determined based on the first of these events. Patients were followed until reaching the above-mentioned composite end point, death (as determined by a linkage to the Manitoba Health Insurance Registry), a maximum of 5 years, or loss to follow-up.

Using laboratory creatinine measurements as described for the Manitoba cohort described previously, 40% decline in eGFR was identified. Kidney failure was defined similarly, but with minor adaptations necessitated by a structurally different administrative data set (see FIG. 30). Chronic dialysis and kidney transplants were identified using the Northern and Southern Alberta Renal Program databases, a provincial registry of renal replacement—any single code for hemodialysis, peritoneal dialysis, or transplant was used. (Note: Because the registry begins in 2001, physician claims data were also used when excluding individuals with prior transplants or dialysis). These data were linked sources to the provincial laboratory repository by unique, encoded, patient identifiers.

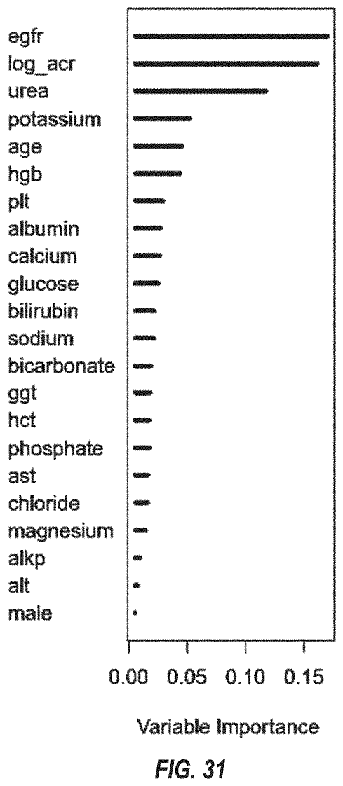

Baseline characteristics for the development (internal training and testing) and external validation cohorts were summarized with descriptive statistics. A random forest model was developed using the R package Fast Unified Random Forest for Survival, Regression, and Classification using a survival forest with right-censored data. Data were split into training (70%) and testing (30%) data sets with a single split and then validated in an external cohort. Models were evaluated for accuracy using the area under the receiver operating characteristic curve, the Brier score, and calibration plots of observed versus predicted risk. Area under the receiver operating characteristic curve and Brier scores were assessed for prediction of the outcome at 1 to 5 years, in 1-year intervals, and calibration plots were evaluated at 2 and 5 years. Model hyperparameters were optimized using the tune.rfsrc function using comparisons of the maximal size of the terminal node and the number of variables to possibly split at each node to the out-of-bag error rate from the Random Forest for Survival, Regression, and Classification package. In addition, sensitivity, specificity, negative predictive value (NPV), and positive predictive value (PPV) were assessed for the top 10%, 15%, and 20% of patients at highest estimated risk (high risk), including for the bottom 50%, 45%, and 30% at lowest risk (low risk). These metrics were assessed at 2 and 5 years. A visualization of the risk of progression versus predicted probability was plotted for 2 and 5 years. Using the final grown 22-variable survival forest, variable importance of included parameters was evaluated, as shown in FIG. 31.

To evaluate robustness, the model was evaluated in subpopulations of the testing and validation cohorts for the 5-year prediction of the primary outcome defined by CKD stage and the presence or absence of diabetes. For sensitivity analyses, 2 comparator models were considered. (i) A Cox proportional hazards model was evaluated using a guideline-based definition of risk using the 3-level definition of albuminuria and 5 stages of eGFR as categorical predictors as a comparator (heatmap model). (ii) A Cox proportional hazards model was evaluated including the variables eGFR, urine ACR, diabetes, hypertension, stroke, myocardial infarction, age, and sex (clinical model). In addition, the model was evaluated in the external validation cohort where laboratory values were only included 1 year before the index date.

Analysis was performed using R Version 4.1.0. Statistical significance was a priori identified using an a ¼ 0.05. For the development cohort (training and testing), a total sample size of 77,196, allocating 54,037 to the training data set (70%) and 23,159 to the testing data set, was used. A total of 321,396 individuals were identified in the validation cohort, with a random subset of 107,097 selected for evaluation. Detailed overview of the cohort selection process for both the development and validation cohorts is provided in FIGS. 5 and 28.

The mean age of the development cohort was 59.3 years, with a mean eGFR of 82.2 ml/min per 1.73 m² and median urine ACR of 1.1 mg/mmol. Of the patients, 48% were male, 45% had diabetes, 70% had hypertension, 5% had a history of congestive heart failure, 4% a prior stroke, and 3% a prior myocardial infarction (similar between the testing and training cohorts).

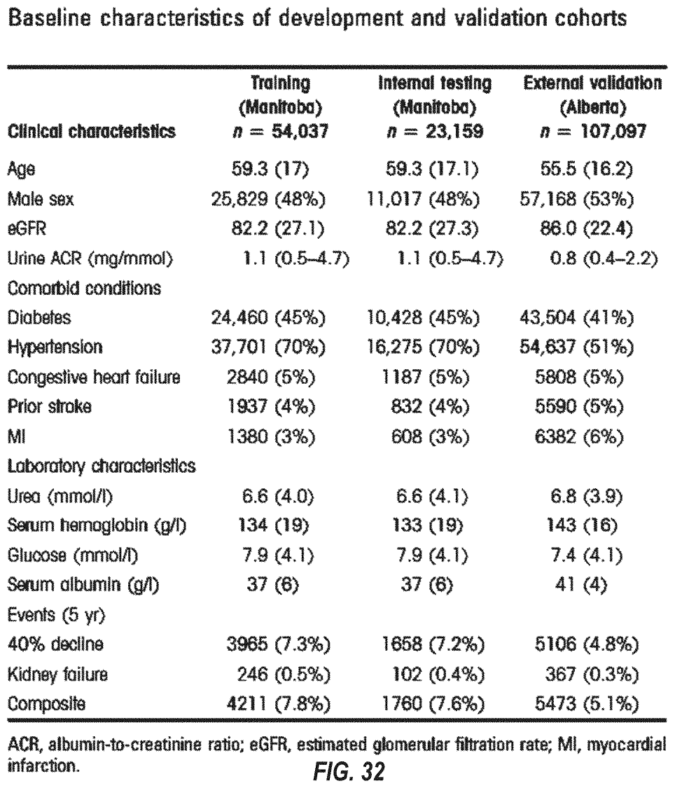

The validation cohort was slightly younger, with a mean age of 55.5 years, mean eGFR of 86.0 ml/min per 1.73 m², and median ACR of 0.8 mg/mmol. The validation cohort had a higher proportion of male patients (53%), 41% of patients had diabetes, 51% hypertension, 5% a history of congestive heart failure, 5% a prior stroke, and 5% a prior myocardial infarction. An overview of baseline descriptive statistics is provided in FIG. 32

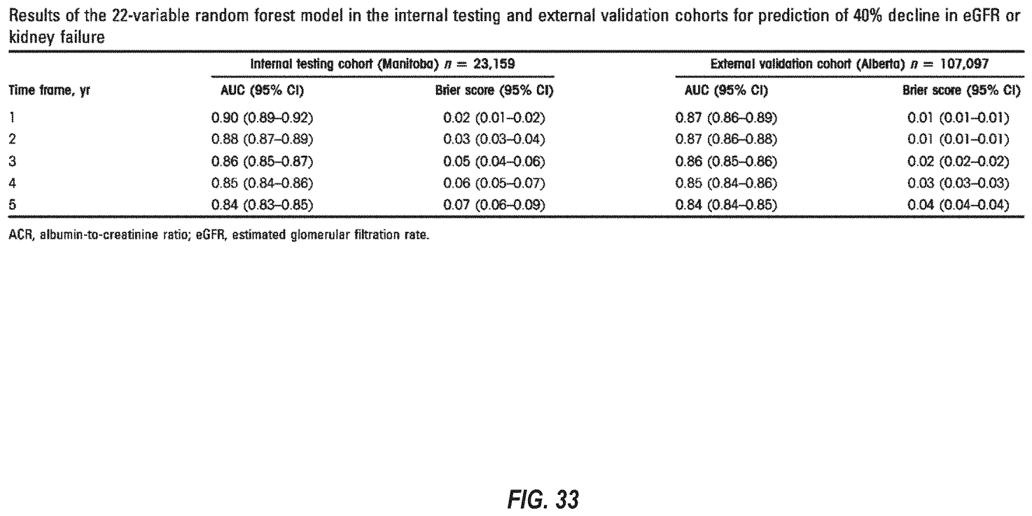

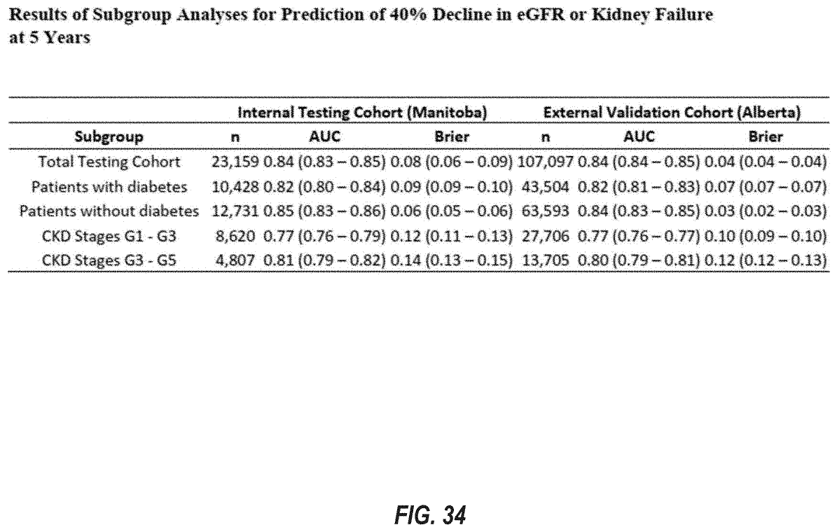

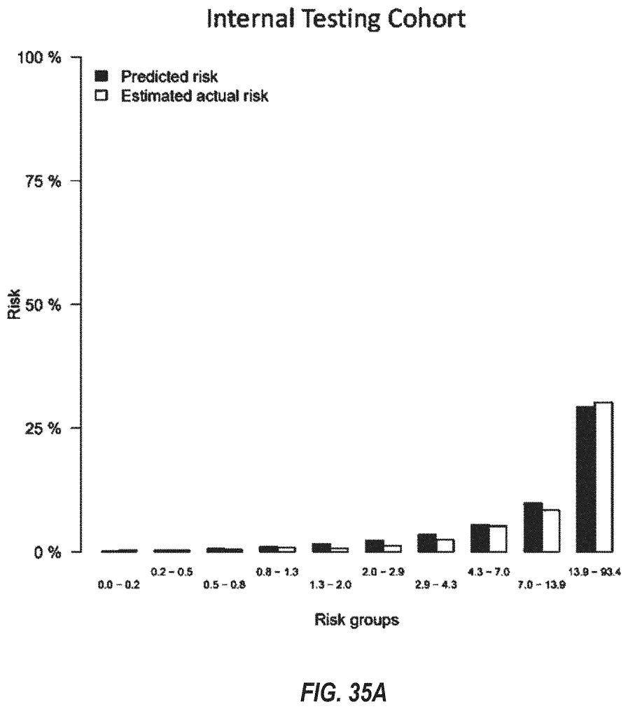

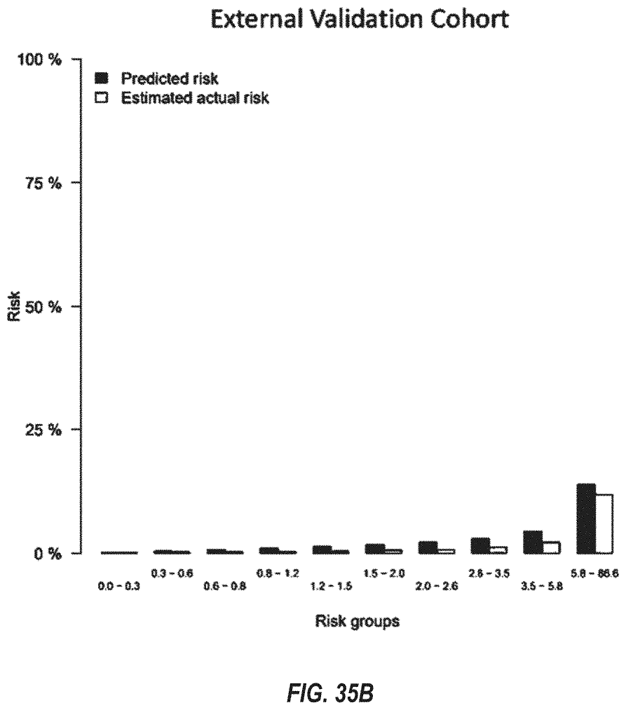

In the random survival forest model with 22 variables, when evaluated in the testing cohort, an AUC of 0.90 (0.89-0.92) for 1-year prediction of the primary outcome and 0.84 (0.83-0.85) for 5-year prediction was found. The Brier score was 0.02 (0.01-0.02) for 1-year prediction of the primary outcome and 0.07 (0.06-0.09) for 5-year prediction. AUCs and Brier scores for years 1 to 5 are presented in FIG. 33. AUC and Brier score were similar in the predefined subgroups (FIG. 34). The model exhibited excellent calibration at both 2 and 5 years (see FIGS. 35A and 35B) in both the internal and external testing cohorts. In addition, a relationship between occurrence of the primary outcome event was observed to increase with increasing predicted probability generated by the random forest algorithm.

Statistics were evaluated on sensitivity, specificity, and PPV in high-risk patients (top 10%, 15%, and 20% of risk scores, respectively). For prediction of the primary outcome at 2 years, it was found that patients in the top decile (14% 2-year risk threshold) had a sensitivity of 58%, a specificity of 92%, and a PPV of 25%. Similarly, for the top 15% of patients (10% 2-year risk threshold), a sensitivity of 69%, specificity of 87%, and PPV of 20% was found. For the top 20% of patients (7% 2-year risk threshold) sensitivity was 76%, specificity was 83%, and PPV was 16%. Using a 30% threshold to identify high- and intermediate-risk patients, 87% of individuals with an event in 2 years and 77% within 5 years would have been identified.

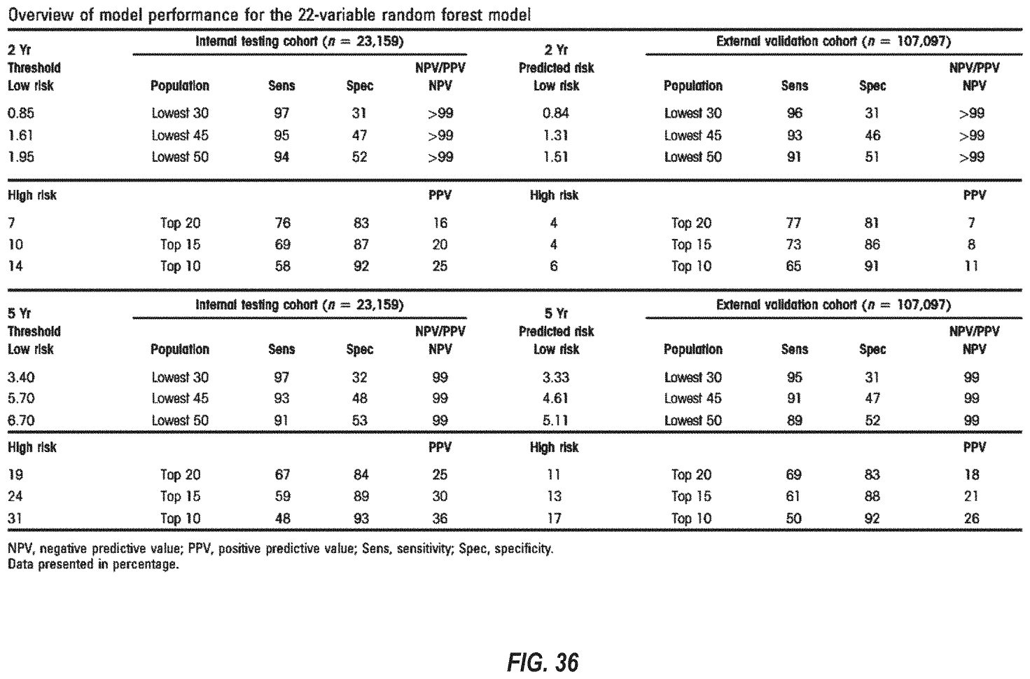

In the low-risk patients, it was found that the bottom 50% of patients (1.95% 2-year risk threshold) had a sensitivity of 94%, specificity of 52%, and NPV of >99%. For the lowest 45% of risk scores (1.61% 2-year risk threshold), sensitivity was 95%, specificity was 47%, and NPV was >99%. Last, for the lowest 30% of risk scores (0.85% 2-year risk threshold), a sensitivity of 97%, a specificity of 31%, and an NPV >99% was found. These statistics were considered for the prediction of the outcome at 5 years and found similar accuracy (see FIG. 36).

Urine ACR (including converted PCRs) was the most influential variable in the random forest model, followed by eGFR, urea, hemoglobin, age, serum albumin, hematocrit, and glucose. As noted above, an overview of model inputs ranked by importance is detailed in FIG. 31.

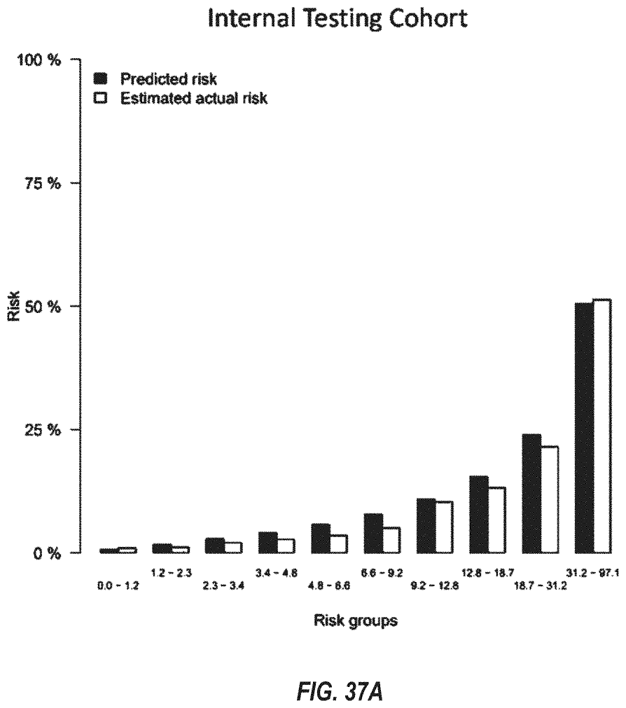

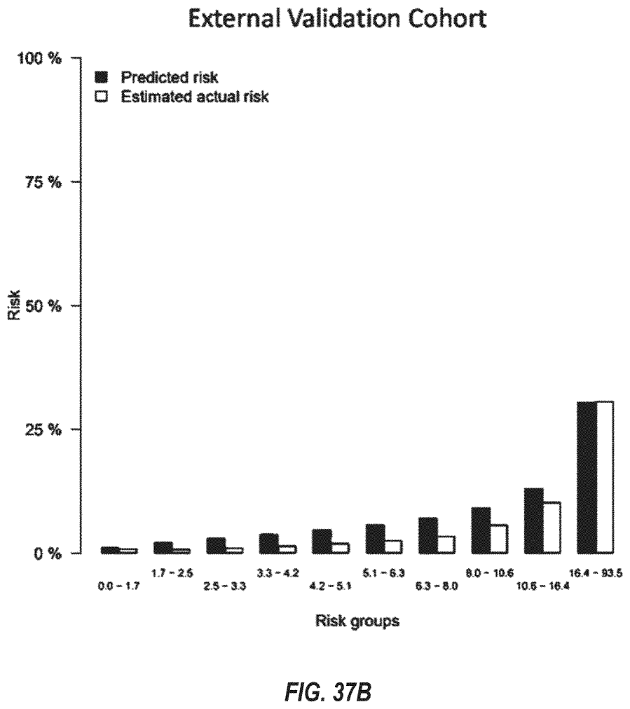

Performance was found to be similar when evaluated in the external validation cohort with an AUC of 0.87 (0.86-0.89) for 1-year prediction declining to 0.84 (0.84-0.85) for 5-year prediction, with Brier scores of 0.01 (0.01-0.01) at 1 year and 0.04 (0.04-0.04) at 5 years (FIG. 33). The external validation cohort had a lower overall risk at both 2 years and 5 years, but the model exhibited excellent calibration (FIGS. 37A and 37B) and a similar increasing association between rank of the risk score and probability of the composite outcome.

In addition, subgroup analyses in patients with and without diabetes, CKD stages G1 to G3, and eGFR <60 ml/min per 1.73 m² had similar outcomes to the internal testing cohort (FIG. 34). Similar diagnostic accuracy, evaluated with sensitivity, specificity, NPV, and PPV, was observed in the external validation cohort as that of the development cohort (FIG. 36).

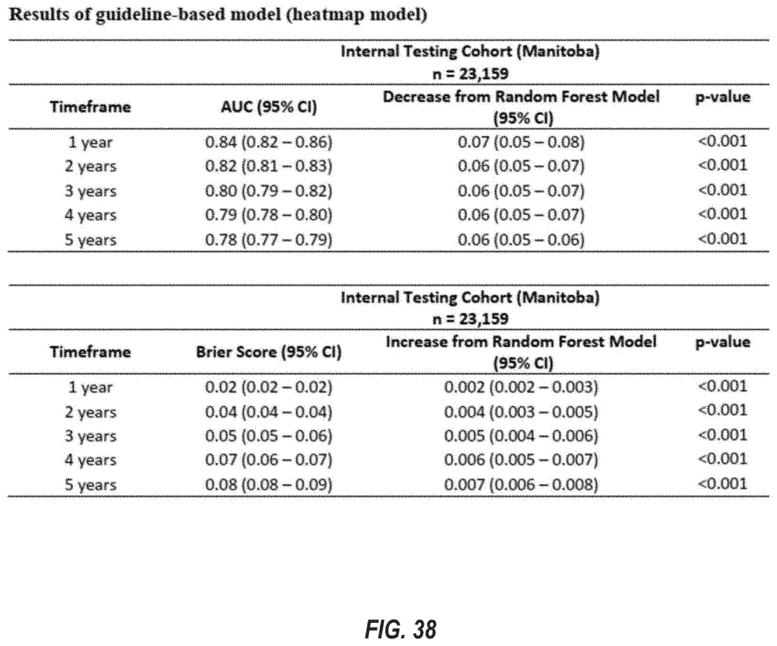

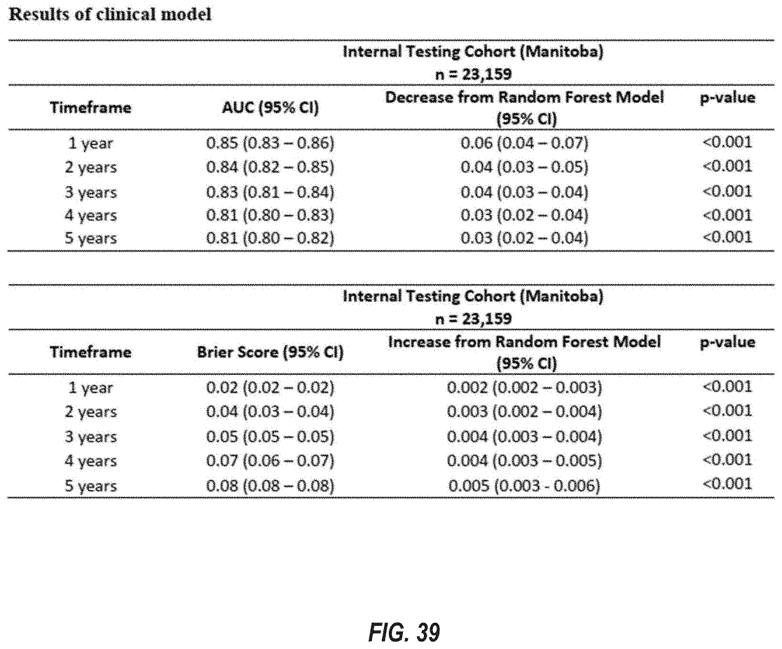

In the comparator analysis, the heatmap model performed worse than the 22-variable random survival forest model in the development cohort (C statistic 0.78 at 5 years vs. 0.84, FIG. 38), as did the clinical model (C statistic 0.81 at 5 years, P<0.001, FIG. 39). When considering only laboratory values in the 12 months preceding the index date, the results of model evaluation for the random forest model were unchanged (1-year AUC of 0.87, 0.86-0.88; 5-year AUC 0.84, 0.83-0.85).

### Conclusion

At least some disclosed embodiments provide externally evaluated laboratory-based prediction models for the outcomes of kidney failure or 40% decline in eGFR. Disclosed models can be entirely based on a single time point measure of routinely collected laboratory data and predict the outcomes of interest (CKD progression) with greater accuracy than current standard of care or commercially available models that test for novel biomarkers and/or attempt to use machine learning methods. Taken together, the models disclosed herein can be implemented in clinical and research settings.

At least some of the disclosed machine learning models using a random forest or random survival forest appear to perform better than commercially available machine learning models, such as RenalytixAI. Compared with the RenalytixAI tool, at least some of the disclosed models have the advantage of having had external validity in an independent population and are therefore at lower risk for overfitting. This step is particularly important for machine learning models which, when derived in small data sets with many predictors, tend to overfit the development population and often do not generalize well. Furthermore, at least some of the disclosed models require only easily mapped laboratory data, which may make them easier to implement at scale than models requiring multiple electronic health record fields and data types, such as the RenalytixAI tool.

Finally, at least some of the disclosed models do not require (and may expressly omit) the measurement or use as input of any novel or proprietary biomarkers, in contrast with RenalytixAI. Therefore, at least some of the disclosed models can be implemented in a routine laboratory setting or using already collected laboratory data.

There are important clinical and research implications of the disclosed models. From a clinical perspective, physicians can use at least some of the disclosed models in office to identify patients who are early in their course of CKD (eGFR >60 ml/min per 1.73 m²), but at high risk of progression in the next 5 years. Given the effect of interventions such as SGLT2 inhibitors on the slope of eGFR in this population, it is possible that these patients may be able to forestall or prevent the lifetime occurrence of kidney failure entirely versus delaying the time to dialysis if the interventions are implemented later in course of disease. In addition, newer therapies such as finerenone may provide additional benefit for slowing CKD progression; however, such newer and/or developing therapies have been largely studied in patients with preserved kidney function and may be initially reserved for intermediate and high risk subgroups to maximize benefit while reducing the burden of cost and polypharmacy. Implementing the disclosed models may facilitate guided use of such newer therapies for at-risk individuals in a targeted, efficient manner.

From a research perspective, several large clinical trials have used 40% decline in eGFR or kidney failure as the primary outcome, and validation of at least some of the disclosed models in those trial data sets may help highlight risk treatment interactions. For future trials that are currently in planning or enrolment phases, the use of at least some of the disclosed models may be helpful to enrich the trial population to generate the appropriate number of outcomes in a reasonable time frame.

At least some strengths of the embodiments discussed hereinabove include external validation, which is particularly important for machine learning models as they can overfit small data sets that have many predictor variables. In addition to this point, it has been found that at least some disclosed models were able to externally validate with high discrimination in a cohort that had total missingness for 2 variables. Additional strengths include novel research methods that include random forest methodology on 2 well described data sets, findings from which have been proven generalizable for multiple kidney outcomes and interventions. A notable strength is the reliance only on routinely collected laboratory data, enabling rapid integration into electronic health records and laboratory information systems.

In conclusion, machine learning models are disclosed that use routinely collected laboratory data and predict CKD progression (40% decline in eGFR or kidney failure) with accuracy for all patients with CKD (e.g., even for patients in early stages of CKD, such as G1 or G2).

### Additional Terms & Definitions

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. Further, elements described in relation to any embodiment depicted and/or described herein may be combinable with elements described in relation to any other embodiment depicted and/or described herein.

The terms “approximately,” “about,” and “substantially” as used herein represent an amount or condition close to the stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a stated amount or condition.

In some embodiments, a time period (or time point or timeframe) refers to a single minute, a single hour, a single day, a single week, or a single year. Alternatively, in some embodiments, a time period refers to a time duration such as over multiple hours, over multiple days, over multiple weeks, or over multiple years, wherein the time period has a first starting time and a second ending time subsequent to the first starting time. Typically, the input data set for a new patient as described herein includes medical laboratory data based on one or more samples obtained from a patient within a single testing period (typically labs ordered from a single physician's visit, or a string of related and/or collective physician's visits which are scheduled to diagnosis and/or treat a particular set of symptoms or a particular disease, for example, CKD).

### Additional Computer System Details

Embodiments of the present invention may comprise or utilize a special purpose or general-purpose computer (e.g., computing system 110) including computer hardware, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system. Computer-readable media (e.g., hardware storage device(s) 140 of FIG. 1) that store computer-executable instructions (e.g., computer-readable instructions 118 of FIG. 1) are physical hardware storage media/devices that exclude transmission media. Computer-readable media that carry computer-executable instructions or computer-readable instructions (e.g., computer-readable instructions 118) in one or more carrier waves or signals are transmission media. Thus, by way of example, and not limitation, embodiments of the invention can comprise at least two distinctly different kinds of computer-readable media: physical computer-readable storage media/devices and transmission computer-readable media.

Physical computer-readable storage media/devices are hardware and include RAM, ROM, EEPROM, CD-ROM or other optical disk storage (such as CDs, DVDs, etc.), magnetic disk storage or other magnetic storage devices, or any other hardware which can be used to store desired program code means in the form of computer-executable instructions or data structures and which can be accessed by a general purpose or special purpose computer.

A “network” (e.g., network 130 of FIG. 1) is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmission media can include a network and/or data links which can be used to carry, or desired program code means in the form of computer-executable instructions or data structures, and which can be accessed by a general purpose or special purpose computer. Combinations of the above are also included within the scope of computer-readable media.

Further, upon reaching various computer system components, program code means in the form of computer-executable instructions or data structures can be transferred automatically from transmission computer-readable media to physical computer-readable storage media (or vice versa). For example, computer-executable instructions or data structures received over a network or data link can be buffered in RAM within a network interface module (e.g., a “NIC”), and then eventually transferred to computer system RAM and/or to less volatile computer-readable physical storage media at a computer system. Thus, computer-readable physical storage media can be included in computer system components that also (or even primarily) utilize transmission media.

Computer-executable instructions comprise, for example, instructions and data which cause a general-purpose computer, special purpose computer, or special purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

What is claimed is:

1. A method, comprising:
accessing a machine learning model configured to generate a prediction of chronic kidney disease (CKD) progression, the machine learning model being trained on a training dataset comprising (i) a first set of medical laboratory data associated with a plurality of patients, (ii) an age of each patient included in the plurality of patients, and (iii) a sex of each patient included in the plurality of patients, the first set of medical laboratory data indicating, for at least a combination of patients included in the plurality of patients: estimated glomerular filtration rate (eGFR), urine albumin-to-creatinine ratio (ACR), urea, serum sodium, serum chloride, serum hemoglobin, serum potassium, glucose, serum albumin, alkaline phosphatase, serum phosphate, serum bicarbonate, serum magnesium, serum calcium, aspartate aminotransferase (AST), alanine transaminase (ALT), bilirubin, gamma-glutamyl transferase (GGT), hematocrit, and platelet count; and
generating a prediction of CKD progression for a new patient by applying an input dataset associated with the new patient to the machine learning model, the prediction of CKD progression for the new patient being based upon output of the machine learning model resulting from applying the input dataset associated with the new patient to the machine learning model, the input dataset comprising an age of the new patient, a sex of the new patient, and a second set of medical laboratory data indicating for the new patient one or more of: eGFR, urine ACR, urea, serum sodium, serum chloride, serum hemoglobin, serum potassium, glucose, serum albumin, alkaline phosphatase (ALKP), serum phosphate, serum bicarbonate, serum magnesium, serum calcium, AST, ALT, bilirubin, GGT, hematocrit, and platelet count.

2. The method of claim 1, wherein the new patient is not associated with a CKD stage of G3 or later.

3. The method of claim 1, wherein the machine learning model comprises a random survival forest model.

4. The method of claim 1, wherein the prediction of CKD progression indicates a risk of experiencing CKD progression within a particular amount of time from a time period associated with the input dataset for the new patient.

5. The method of claim 4, wherein the particular amount of time is provided as input to the machine learning model for generating the prediction of CKD progression.

6. The method of claim 4, wherein the particular amount of time comprises 2 years or 5 years.

7. The method of claim 1, wherein the urine ACR for one or more of the plurality of patients or the new patient is converted from a urine protein-to-creatinine test or a urine dipstick test.

8. The method of claim 1, wherein the prediction of CKD progression comprises a prediction of a risk of the new patient experiencing kidney failure or about a 40% or greater decline of the eGFR for the new patient.

9. The method of claim 8, wherein the risk of kidney failure comprises an indication that the new patient is at risk of (i) requiring chronic dialysis, (ii) requiring a kidney transplant, or (iii) experiencing a glomerular filtration rate of less than 10 ml/min/1.73 m².

10. The method of claim 1, further comprising:
determining that the prediction of CKD progression indicates a predicted risk of the new patient experiencing CKD within a particular time period that satisfies one or more predicted risk threshold values; and
(i) generating a notification that the new patient may need an interventive kidney treatment;
(ii) generating a recommendation of an interventive kidney treatment for the new patient based on the prediction of CKD progression;
(iii) generating a recommendation of a frequency of monitoring of CKD progression for the new patient based on the prediction of CKD progression; or
(iv) administering an interventive kidney treatment to the new patient.

11. The method of claim 10, wherein the one or more predicted risk threshold values are based upon the particular time period associated with the prediction of CKD progression.

12. The method of claim 10, wherein the recommendation of the interventive kidney treatment or the recommendation of the frequency of monitoring of CKD progression is further based upon at least some of the second set of medical laboratory data associated with the new patient.

13. The method of claim 10, wherein the interventive kidney treatment comprises one or more of: renin-angiotensin-aldosterone system (RAAS) inhibition, blood pressure control, sodium-glucose cotransporter-2 (SGLT2) inhibitor medication, mineralocorticoid receptor antagonists (MRAs) therapy, or preparation for nephrology consultation, home dialysis, dialysis access, or kidney transplant.

14. The method of claim 1, wherein the first set of medical laboratory data comprises one or more imputed values in place of missing values.

15. The method of claim 14, wherein the first set of medical laboratory data indicates, with a degree of value imputation of 30% or less, eGFR, urine ACR, urea, potassium, hemoglobin, platelet count, albumin, calcium, glucose, bilirubin, sodium, bicarbonate, and GGT.

16. A system, comprising:
one or more processors; and
one or more hardware storage devices storing instructions that are executable by the one or more processors to configure the system to:
access a training dataset comprising (i) a first set of medical laboratory data associated with a plurality of patients, (ii) an age of each patient included in the plurality of patients, and (iii) a sex of each patient included in the plurality of patients, the first set of medical laboratory data indicating, for at least a combination of patients included in the plurality of patients: estimated glomerular filtration rate (eGFR), urine albumin-to-creatinine ratio (ACR), urea, serum sodium, serum chloride, serum hemoglobin, serum potassium, glucose, serum albumin, alkaline phosphatase, serum phosphate, serum bicarbonate, serum magnesium, serum calcium, aspartate aminotransferase (AST), alanine transaminase (ALT), bilirubin, gamma-glutamyl transferase (GGT), hematocrit, and platelet count; and
generate a machine learning model by applying the training dataset to an untrained model, the machine learning model being configured to generate a prediction of chronic kidney disease (CKD) progression for a new patient by applying an input dataset associated with the new patient to the machine learning model, the input dataset comprising an age of the new patient, a sex of the new patient, and a second set of medical laboratory data indicating for the new patient one or more of: eGFR, urine ACR, urea, serum sodium, serum chloride, serum hemoglobin, serum potassium, glucose, serum albumin, alkaline phosphatase (ALKP), serum phosphate, serum bicarbonate, serum magnesium, serum calcium, AST, ALT, bilirubin, GGT, hematocrit, and platelet count.

17. The system of claim 16, wherein the machine learning model comprises a random survival forest model.

18. One or more hardware storage devices storing instructions that are executable by one or more processors of a system to configure the system to:
access a machine learning model configured to generate a prediction of chronic kidney disease (CKD) progression, the machine learning model being trained on a training dataset comprising (i) a first set of medical laboratory data associated with a plurality of patients, (ii) an age of each patient included in the plurality of patients, and (iii) a sex of each patient included in the plurality of patients, the first set of medical laboratory data indicating, for at least a combination of patients included in the plurality of patients: urine albumin-to-creatinine ratio (ACR), estimated glomerular filtration rate (eGFR), urea, hemoglobin; and
generate a prediction of CKD progression for a new patient by applying an input dataset associated with the new patient to the machine learning model, the prediction of CKD progression for the new patient being based upon output of the machine learning model resulting from applying the input dataset associated with the new patient to the machine learning model, the input dataset comprising an age of the new patient, a sex of the new patient, and a second set of medical laboratory data comprising one or more components of a urine chemistry test, a comprehensive metabolic panel, a complete blood cell count, a liver panel, or a uric acid test for the new patient.

19. The one or more hardware storage devices of claim 18, wherein the second set of medical laboratory data comprises one or more components of the urine chemistry test for the new patient.

20. The one or more hardware storage devices of claim 19, wherein the second set of medical laboratory data comprises one or more components of the urine chemistry test and the comprehensive metabolic panel for the new patient.
